(19) United States
(12) Patent Application Publication
(10) Pub. No.: US 2024/0196873 A1
(43) Pub. Date: Jun. 20, 2024

(54) SYSTEM AND METHOD FOR REARING A GENETICALLY-MODIFIED FLY POPULATION TO REGULATE PRODUCTION OF A TARGET COMPOUND

(71) Applicant: Future Fields Cellular Agriculture and Research LTD., Edmonton (CA)

(72) Inventors: Erik Jason Luber, Edmonton (CA); Brian Charles Olsen, Edmonton (CA)

(21) Appl. No.: 18/541,697

(22) Filed: Dec. 15, 2023

Related U.S. Application Data
(60) Provisional application No. 63/432,946, filed on Dec. 15, 2022.

(51) Int. Cl. A01K 67/033

(52) U.S. Cl. CPC A01K 67/0339; A01K 2217/03; A01K 2227/706

(57) ABSTRACT

One variation of a method includes: at a feeder module, subjecting a first set of cages to a first set of environmental conditions configured to promote deposition of fly eggs by adult flies, each cage, in the first set of cages, containing a fly population, in a set of fly populations, genetically modified to generate amounts of a target compound; at an incubator module, subjecting a second set of cages to a second set of environmental conditions configured to promote transformation of fly eggs into fly larvae; at a treatment module, subjecting a third set of cages to a third set of environmental conditions configured to promote generation of a target compound in fly larvae contained within the third set of cages; and, at a repopulation module, subjecting a fourth set of cages to a fourth set of environmental conditions configured to promote transformation of fly larvae into adult flies.

### CROSS-REFERENCE TO RELATED APPLICATIONS

This application claims the benefit of U.S. Provisional Application No. 63/432,946, filed on 15 Dec. 2022, which is incorporated in its entirety by this reference.

### TECHNICAL FIELD

This invention relates generally to the field of transgenics and more specifically to a new and useful method for generating recombinant proteins in the field of transgenics.

### BRIEF DESCRIPTION OF THE FIGURES

FIG. 1 is a flowchart representation of a method;

FIG. 2 is a flowchart representation of one variation of the method;

FIG. 3 is a flowchart representation of one variation of the method;

FIG. 4 is a flowchart representation of one variation of the method;

FIGS. 5A, 5B, and 5C are flowchart representations of one variation of the method; and

FIG. 6 is a flowchart representation of one variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

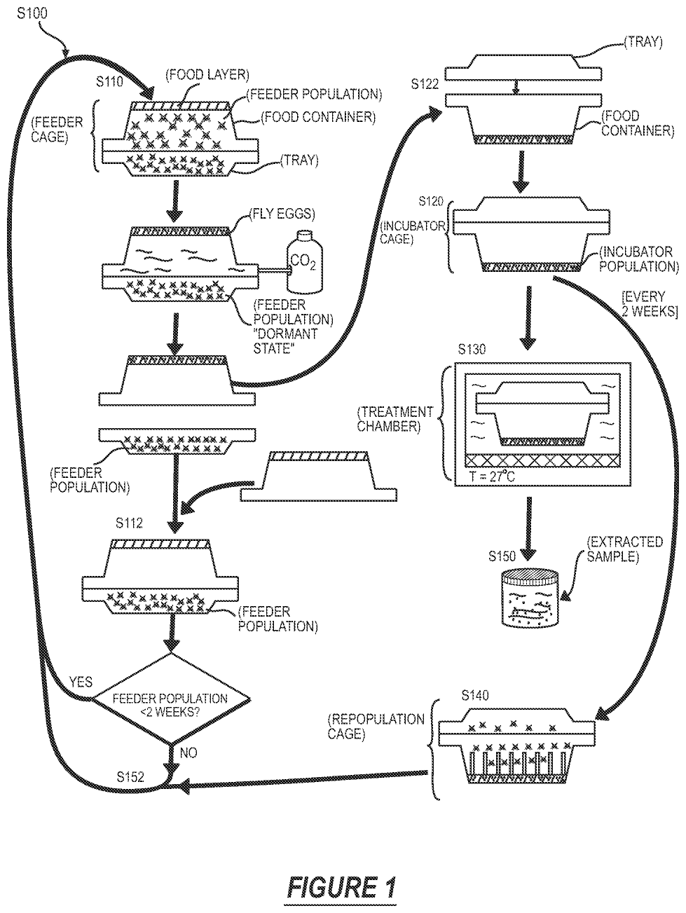

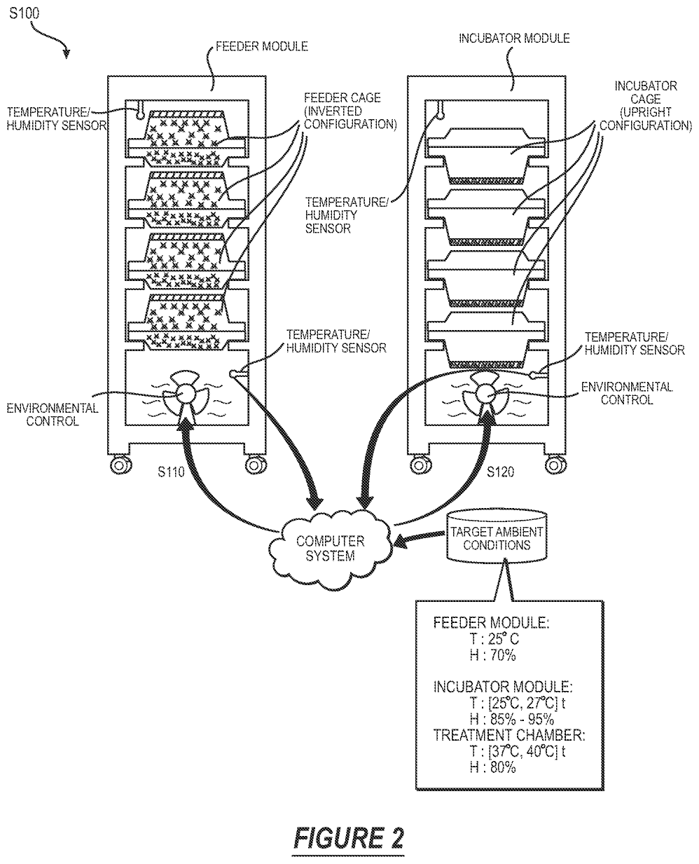

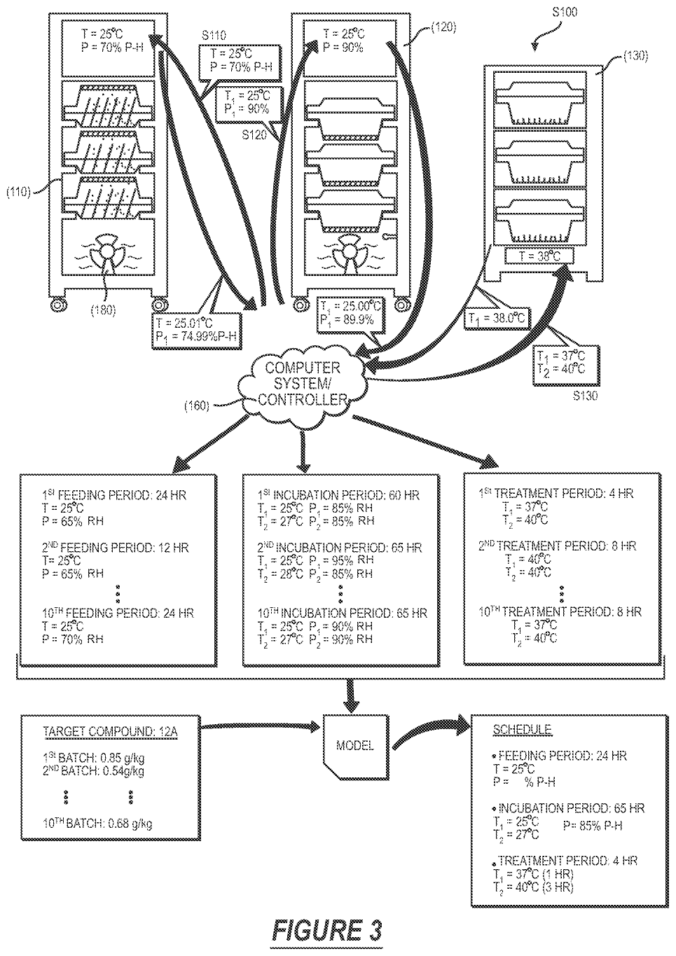

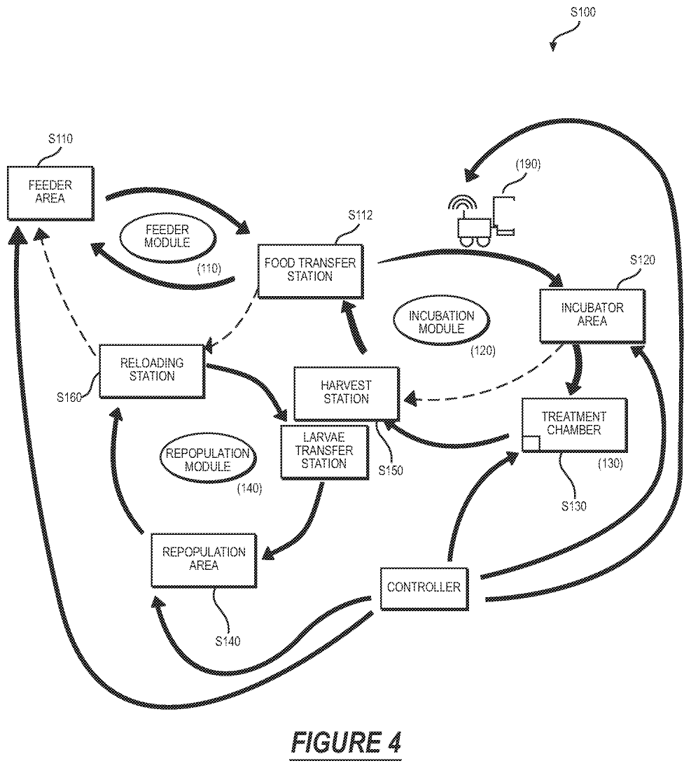

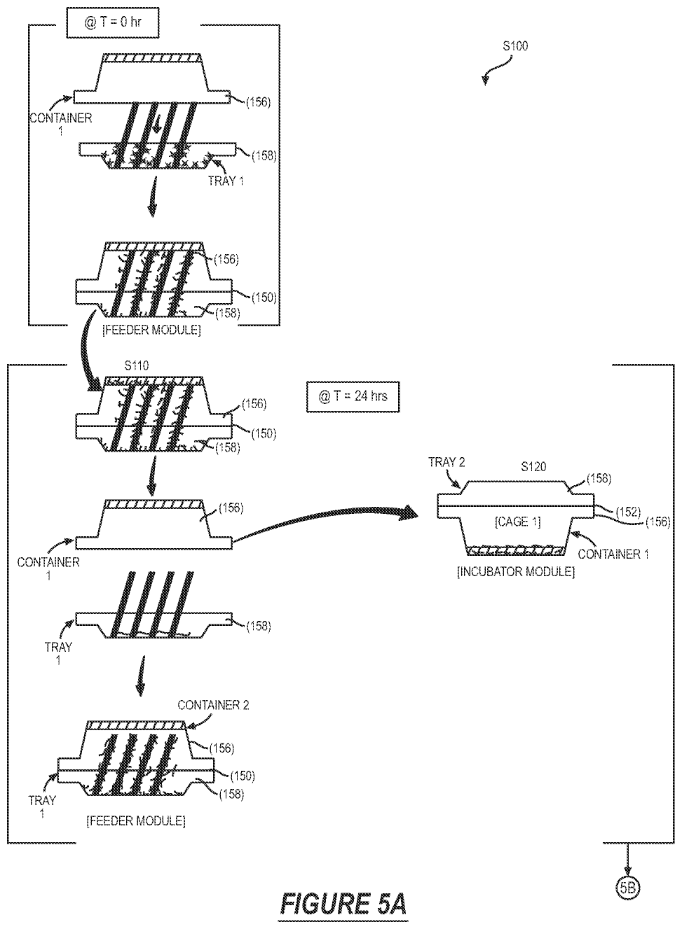

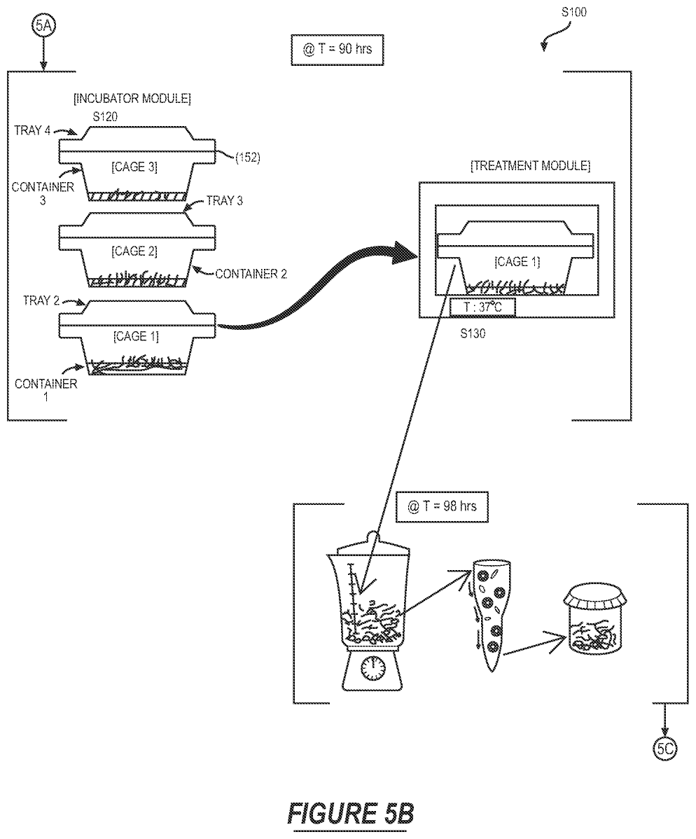

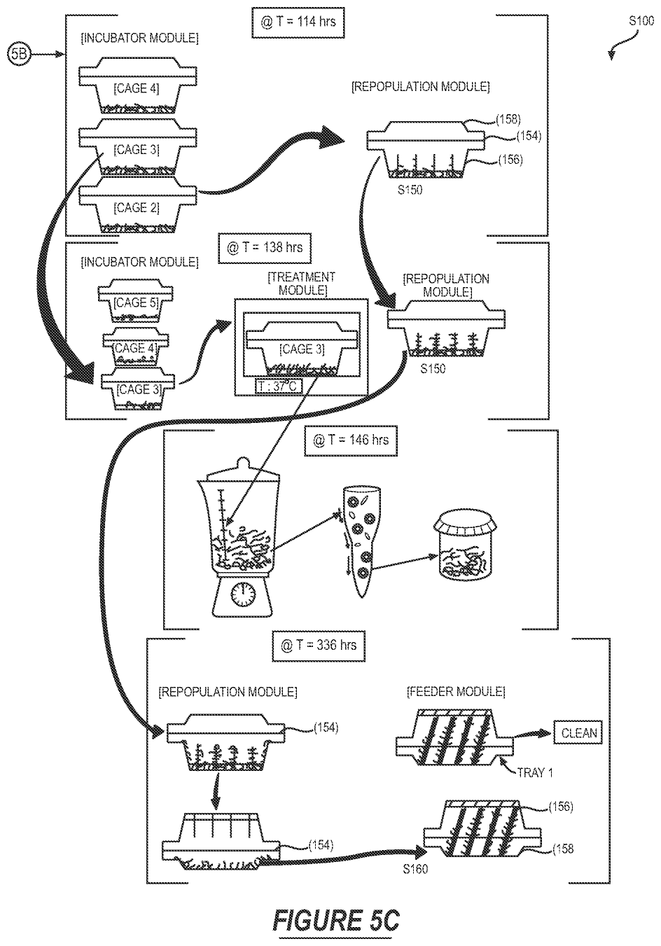

As shown in FIGS. 1-4, 5A-5C, and 6, a method S100 includes, during a first feeding period of a first fixed duration: at a first time, coupling a first container to a first tray 158, occupied by a first population of adult flies, to form a first cage, in a first set of cages, arranged in an inverted configuration and configured to contain the first population of adult flies in Block S112, the first container loaded with a layer of foodstuff facing and configured to seat over the first tray 158 with the layer of foodstuff facing an interior volume of the first tray 158 in the inverted configuration, the first population of adult flies genetically modified to produce amounts of a first target compound; subjecting the first set of cages, arranged within a feeder module 110, to a first set of conditions configured to promote deposition of fly eggs by adult flies in Block S110; and, at a second time succeeding the first time by the first fixed duration, decoupling the first container from the first tray 158 to collect a first population of eggs deposited onto the first container by the first population of adult flies during the first feeding period. The method S100 further includes, during a first incubation period, succeeding the first feeding period, of a second fixed duration: at a third time, coupling the first container to a second tray 158 to form a second cage, in a second set of cages, arranged in an upright configuration, the second tray 158 configured to seat over the first container in the upright configuration and the first container including the first population of eggs facing an interior volume of the second tray 158 at the third time; subjecting the second set of cages, arranged within an incubation module, to a second set of conditions configured to promote transformation of fly eggs into fly larvae in Block S120; and, in response to expiration of the second fixed duration, during a first treatment period succeeding the first incubation period and of a third fixed duration, locating the second cage in a treatment module 130 configured to promote generation of the target compound in fly larvae in Block S130, the second cage containing a first population of larvae grown from the first population of eggs during the first incubation period. The method S100 further includes, in response to expiration of the third fixed duration: harvesting the first population of larvae from the second cage and extracting an amount of the first target compound from the first population of larvae in Block S150.

One variation of the method S100 includes: at a first time, coupling a first container to a first tray 158, occupied by a first population of adult flies, to form a first cage, in the first set of cages, arranged in an inverted configuration and configured to contain the first population of adult flies, the first container loaded with a layer of foodstuff facing and configured to seat over the first tray 158 with the layer of foodstuff facing an interior volume of the first tray 158 in the inverted configuration in Block S112, the first population of adult flies genetically modified to produce amounts of a first target compound; and, during a first feeding period of a first fixed duration and succeeding the first time, subjecting the first cage to a first set of conditions configured to promote deposition of eggs by adult flies in Block S110. in this variation, the method S100 further includes, in response to expiration of the first fixed duration: at a third time, decoupling the first container from the first tray 158 to collect a first population of eggs deposited onto the first container by the first population of adult flies during the first feeding period; and, at a fourth time succeeding the third time, coupling the first container to a second tray 158 to form a second cage arranged in an upright configuration, the second tray 158 configured to seat over the first container in the upright configuration and the first container including the first population of eggs facing an interior volume of the second tray 158 at the third time. The method S100 further includes: during a first incubation period succeeding the fourth time and of a second fixed duration, subjecting the second cage to a second set of conditions configured to promote transformation of the first population of eggs into a first population of larvae in Block S120; in response to expiration of the second fixed duration, during a first treatment period succeeding the first incubation period and of a third fixed duration, locating the second cage in a treatment chamber configured to promote generation of the target compound in Block S130; and, in response to expiration of the third fixed duration, harvesting the first population of larvae from the second cage and extracting an amount of the first target compound from the first population of larvae in Block S150.

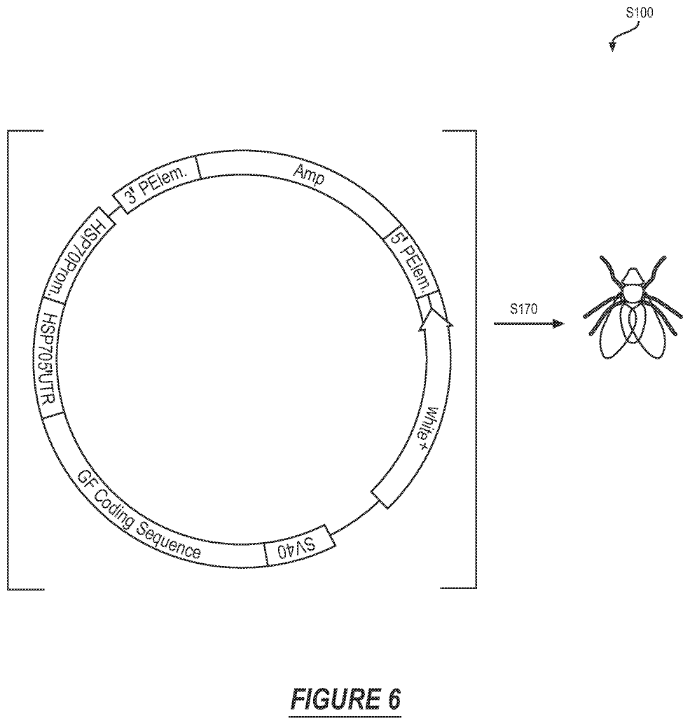

In one variation, as shown in FIG. 6, the method S100 further includes, during an initial time period preceding the first feeding period, genetically modifying a genome of the first population of adult flies to include: a promoter sequence; and a target sequence encoding for the first target compound and linked to the promoter sequence in Block S170.

One variation of the method S100 includes: at a feeder module 110 defining a first array of cage slots, subjecting a first set of cages, transiently arranged within a first array of cage slots, to a first set of environmental conditions configured to promote deposition of fly eggs by adult flies, each cage, in the first set of cages, containing a fly population, in a set of fly populations, genetically modified to generate amounts of a target compound in Block S110; at an incubator module 120 defining a second array of cage slots, subjecting a second set of cages, transiently arranged within second array of cage slots, to a second set of environmental conditions configured to promote transformation of fly eggs into fly larvae in Block S120; at a treatment module 130 defining a third array of cage slots, subjecting a third set of cages, transiently arranged within the third array of cage slots, to a third set of environmental conditions configured to promote generation of a target compound in fly larvae contained within the third set of cages in Block S130; and, at a repopulation module 140 defining a fourth array of cage slots, subjecting a fourth set of cages, transiently arranged within the fourth array of cage slots, to a fourth set of environmental conditions configured to promote transformation of fly larvae into adult flies in Block S140. In this variation, the method S100 further includes—during a global period of a first target duration—during a first cycle: at a first time, transferring a first population of eggs, deposited by a first fly population in a first cage in the first set of cages, from the first cage to a second cage, in the second set of cages, in the incubator module 120; at a second time succeeding the first time by a second target duration, transferring the second cage from the incubator module 120 to the treatment module 130, the second cage containing a first population of larvae, emerged from the first population of eggs, at the second time; and, at a third time succeeding the second time by a third target duration, harvesting the first population of larvae from the second cage for extraction of an amount of the target compound. During the global period, the method S100 further includes, during a second cycle: at a fourth time, transferring a second population of eggs, deposited by the first fly population in the first cage, from the first cage to a third cage, in the second set of cages, in the incubator module 120; at a fifth time succeeding the first time by the second target duration, in response to expiration of the first target duration, transferring the third cage from the incubator module 120 to the repopulation module 140, the third cage containing a second population of larvae, emerged from the second population of eggs, at the fifth time; and, at a sixth time succeeding the fifth time by a fourth target duration, harvesting the first fly population from the first cage and depositing a second fly population—grown from the second population of larvae in the repopulation module 140—in the first cage in the feeder module 110 in Block S160.

In one variation, the method S100 for regulating generation of a target compound includes, during a global period, of a first target duration, for a first fly population: cultivating the fly population in a feeder cage 150—arranged in an inverted configuration within a cage slot, in a first array of cage slots, in a feeder module 110—according to a first set of target ambient conditions defined for the feeder module 110 in Block S110, the feeder cage 150 formed of a first tray 158 and a food container 156 removably coupled to the first tray 158, configured to cooperate with the first tray 158 to retain the first fly population within an inner volume formed by walls of the first tray 158 and the food container 156, and including a layer of foodstuff configured for consumption and deposition of eggs by flies in the fly population; and—at a first target frequency during the global period—triggering release of a volume of gas into the feeder cage 150 to anesthetize the first fly population and locate the first fly population on a bottom surface (e.g., defined by the first tray 158) of the feeder cage 150; decoupling the food container 156 from the first tray 158; coupling a new food container 156 to the first tray 158—containing the first fly population—to reassemble the feeder cage 150 in Block S112; and locating the feeder cage 150 within the cage slot in the feeder module 110.

In this variation, the method S100 further includes, in response to decoupling the food container 156 from the first tray 158: assembling an incubator cage 152 including the food container 156 coupled to a second tray 158, the food container 156 including a remainder of the layer of foodstuff and a population of fly eggs deposited in the layer of foodstuff by the first fly population; and locating the second cage in an upright configuration within a second cage slot, in a second array of cage slots, within an incubator module 120, the layer of foodstuff defining the bottom surface of the second cage in the upright configuration.

In this variation, the method S100 further includes: during an incubation period of a second target duration, cultivating an incubator population—including the population of eggs and/or a population of larvae hatched from the population of eggs—contained within the second cage in the second cage slot, according to a first set of ambient conditions, defined for the incubation module in Block S120; in response to expiration of the second target duration, during a treatment period of a third target duration, locating the second cage in a treatment chamber configured to subject the incubator population to a series of heat-shock cycles configured to induce generation of the target compound in the incubator population in Block S130; and, in response to expiration of the third target duration, harvesting the incubator population from the second cage and extracting an amount of the target compound from the incubator population in Block S150.

The method S100 further includes, during the global period, cultivating a replacement population—including larvae and/or adult flies harvested from the incubator population at a fixed frequency—in a repopulation cage 154 within a repopulation module 140 according to a third set of ambient conditions defined for the repopulation module 140 in Block S140. In response to expiration of the first target duration of the global period, the method S100 further includes: transferring the replacement population from the repopulation cage 154 to a second feeder cage 150; and locating the second feeder cage 150 within the first cage slot in the feeder module 110 in replacement of the first feeder cage 150 in Block S160.

### 2. System

As shown in FIGS. 1-4, 5A, 5B, and 5C, a system 100 includes a set of modules including: a feeder module 110 transiently arranged in a first region of a facility and defining a first array of cage slots assigned to feeder cages 150 containing adult fly populations genetically modified to generate amounts of a first target compound; an incubator module 120 transiently arranged in a second region of the facility and defining a second array of cage slots configured to receive incubator cages 152 containing populations of fly eggs configured to transform into populations of fly larvae; a treatment module 130 transiently arranged in a third region of the facility and defining a third array of cage slots configured to receive incubator cages 152 containing populations of fly larvae; and a repopulation module 140 transiently arranged in a fourth region of the facility and defining a fourth array of cage slots configured to receive repopulation cages 154 containing populations of fly larvae configured to transform into populations of adult flies. The system 100 further includes: a suite of sensors configured to output signals representing environmental conditions at the set of modules and including a first set of sensors 170 coupled to the feeder module 110, a second set of sensors 170 coupled to the incubator module 120, a third set of sensors 170 coupled to the treatment module 130, and a fourth set of sensors 170 coupled to the repopulation module 140; a suite of environmental controls configured to regulate environmental conditions within the facility and including a first set of environmental controls 180 installed in the first region, a second set of environmental controls 180 installed in the second region, a third set of environmental controls 180 installed in the third region, and a fourth set of environmental controls 180 installed in the fourth region; and a set of robotic manipulators 190 configured to transiently retrieve cages from the set of modules, transfer cages between modules in the set of modules, and locate cages within the set of modules.

The system 100 further includes a controller 160 configured to: coordinate motion of the set of robotic manipulators 190 according to a schedule defined for fly populations genetically modified to generate the first target compound; based on signals output by the first set of sensors 170, selectively trigger actuation of the first set of environmental controls 180 to regulate environmental conditions at the feeder module 110 according to a target set of feeder conditions defined for the feeder module 110 and configured to promote deposition of eggs by adult flies in feeder cages 150; based on signals output by the second set of sensors 170, selectively trigger actuation of the second set of environmental controls 180 to regulate environmental conditions at the incubator module 120 according to a target set of incubator conditions defined for the incubator module 120 and configured to promote transformation of fly eggs into fly larvae in incubator cages 152; based on signals output by the third set of sensors 170, selectively trigger actuation of the third set of environmental controls 180 to regulate environmental conditions at the treatment module 130 according to a target set of treatment conditions defined for the treatment module 130 and configured to promote generation of the first target compound in fly larvae; and, based on signals output by the fourth set of sensors 170, selectively trigger actuation of the fourth set of environmental controls 180 to regulate environmental conditions at the repopulation module 140 according to a target set of growth conditions defined for the repopulation module 140 and configured to promote growth of fly larvae into adult flies.

### 3. Applications

Generally, Blocks of the method S100 can be executed to: cultivate a genetically-modified, adult fly population—configured to generate a recombinant protein (hereinafter “target compound”)—within a fly rearing module; to regularly (e.g., at a fixed or variable frequency) collect batches of fly eggs deposited by the adult fly population in the fly rearing module; to facilitate growth of a fly larvae population—derived from batches of fly eggs deposited by the fly population—within a larvae rearing module; regulate generation of the target compound in fly larvae populations by subjecting fly larvae to controlled heat-shock treatment cycles; harvest these fly larvae populations to extract batches of the target compound; and—in response to expiration of a target life cycle for the adult fly population (e.g., housed within the fly rearing module)—replace the current adult fly population with a subsequent adult fly population derived from a batch of fly eggs deposited by the current adult fly population (e.g., within the fly rearing module) and reserved for repopulating the adult fly population.

In particular, a system 100—for rearing fly populations, at various stages of a fly life-cycle, to generate batches of a target compound—can include: a set of modules configured to transiently house cages containing discrete populations of adult flies (hereinafter “feeder populations”) or populations of fly larvae (hereinafter “incubator populations”); and a set of environmental controls 180—such as including temperature, humidity, pressure, and/or air flow regulators—configured to regulate ambient conditions at each module, in the set of modules, according to a set of target ambient conditions defined for the module. The system 100 can also include a set of reconfigurable cages—loaded with foodstuff consumable by flies and/or fly larvae—configured to prevent escape of flies or larvae from these cages and to provide a sustainable living environment for the fly and larvae populations.

Adult fly populations and larvae populations—derived from the adult fly populations—can therefore be cultivated (or “reared”) in these discrete, environmentally-controlled modules. For example, the system 100 can include: an adult-fly rearing module defining a first array of cage slots loaded with a first set of feeder cages 150—each containing a population of adult flies—arranged in an inverted configuration; a larvae rearing module defining a second array of cage slots loaded with a second set of incubator cages 152—each containing a larvae population—arranged in an upright configuration; and a fly repopulation module 140 defining a third array of cage slots loaded with a third set of repopulation cages 154—each containing a developing fly population (e.g., maturing from larvae, into pupae, and into adult flies)—arranged in the upright configuration.

In the preceding example, these modules can be redistributed throughout the facility—such as automatically by an automated mover and/or manually by a user —to enable: transferring of batches of eggs—deposited in foodstuff by the adult-fly population—from adult-fly cages retrieved from the adult-fly rearing module to larvae cages in the larvae rearing module; transferring of larvae cages from the larvae rearing module to a treatment chamber configured to promote generation of the target compound; harvesting of matured larvae from these larvae cages for extraction of the target compound; and/or transferring of matured larvae form these larvae cages into repopulation cages 154 in order to reseed the adult fly population.

### 4. Genetically-Modified Fly Population

Each fly population can be genetically modified to produce a particular target compound (e.g., a growth factor, a Cholera toxin B protein, a collagen protein, insulin). In particular, a genome of flies in the fly population can be genetically modified to include a target sequence (e.g., a DNA sequence) encoding for the target compound, such that when the target sequence is expressed, the insect cells generate the target compound.

In one variation, Blocks of the method S100 can be executed to cultivate multiple adult fly populations, each fly population configured to produce amounts of a particular target compound in a set of target compounds.

In one implementation, a first fly population can be genetically modified to produce amounts of a first target compound, in a set of target compounds, and a second fly population can be genetically modified to produce amounts of a second target compound in the set of target compounds. In particular, in this implementation, the first fly population can be genetically modified to include a first target sequence encoding for the first target compound, such that expression of the first target sequence triggers production of the first target compound in flies in the first fly population. The second fly population can be genetically modified to include a second target sequence encoding for the second target compound, such that expression of the second target sequence triggers production of the second target compound in flies in the second fly population. In one example, Blocks of the method S100 can be executed to: cultivate a first fly population configured to produce amounts of a first protein corresponding to a growth factor; and cultivate a second fly population configured to produce amounts of a second protein corresponding to a therapeutic protein (e.g., insulin, prolactin).

### 4.1 Regulatory Sequences: Expression of the Target Sequence

In one implementation, the genome can be genetically modified to include a set of regulatory sequences—upstream the target sequence—configured to regulate expression of the target sequence and therefore regulate production of the target compound expressed by the target sequence. The set of regulatory sequences can include a promoter sequence coupled to the target sequence. The promoter sequence can be associated with a particular stressor (e.g., heat-shock, cold-shock, nutrient-deprivation, dehydration, UV exposure), such that application of the particular stressor to the fly population activates expression of the promoter sequence.

For example, the genome of the fly population can be genetically modified to include an HSP70 promoter sequence upstream the target sequence, such that activation of the HSP70 promoter sequence leads to expression of the target sequence and thereby generation of the target compound. The HSP70 promoter sequence can activate in the presence of a heat-shock stressor in order to express heat-shock proteins, which may protect cells from damage caused by the heat-shock stressor. More specifically, in response to a particular dosage of a heat-shock stressor, the HSP70 promoter sequence—bound by a set of transcription factors—can initiate transcription of sequences immediately downstream the HSP70 promoter sequence. Therefore, by coupling the target sequence to the HSP70 promoter sequence immediately upstream the target sequence, a heat-shock stressor can be implemented to control transcription of the target sequence and therefore regulate production of the target compound encoded by the target sequence.

### 4.2 Detectable Signal Linked to Target Compound Generation

In one variation, the fly population is genetically modified to generate a detectable signal (e.g., an optical signal) indicating production of the target compound by the population (e.g., when stressed). In particular, cells of the fly population can be genetically modified to include a reporter sequence linked to the promoter sequence and/or the target sequence, such that expression of the reporter sequence is linked to expression of the target sequence in the population. Thus, expression of the reporter sequence by insects in the population can be configured to generate a signal (fluorescence, bioluminescence, and/or pigmentation change) that is directly detectable (e.g., with optical sensors or the human eye) and interpretable as an amount of the target compound present in the fly population without necessitating harvesting and/or chemical testing of the fly population. More specifically, in this variation, the fly population are genetically modified to include a reporter sequence linked to the promoter sequence and/or to the target sequence to enable direct tracking of composition of the target compound within the fly population before, during, and after exposure to a stressor—and prior to harvesting the fly population—based on expression of the reporter sequence (e.g., presence and/or magnitude of a signal generated by the reporter sequence) within the fly population.

For example, the genome of the fly population can be genetically modified to include: a target sequence encoding for a particular target compound; a promoter sequence coupled to the target sequence; and a reporter sequence coupled to the promoter sequence. The reporter sequence can be configured to encode for a fluorescent protein, such that when the reporter sequence is expressed, a detectable fluorescent signal is generated. Therefore, a user (e.g., a lab technician, a researcher) may monitor fluorescence of insects in the fly population to confirm production of the particular target compound.

Further, in this variation, the user may estimate or measure (e.g., via a spectrometer) a magnitude of the signal to estimate an amount (e.g., quantity, proportion, concentration) of the particular target compound within the fly population. In the preceding example, the user may record an intensity of fluorescence generated by the fly population via a handheld spectrometer. This intensity measurement can be leveraged to estimate an amount (e.g., quantity, proportion, concentration) of the particular target compound within the fly population, and the fly population can be continuously exposed or re-exposed to the stressor until the intensity of fluorescence of the fly population reaches an intensity of fluorescence corresponding to a target amount (e.g., exceeding a threshold amount) of the particular target compound.

Therefore, in this variation, by pairing the promoter sequence with a reporter sequence configured to generate a detectable signal when expressed, production of the target compound can be readily confirmed. Furthermore, in this variation, in order to increase magnitude of the secondary signal—and thus production of the target compound—up to a target signal magnitude that corresponds to a target proportion of the target compound within the fly population, the fly population can be: exposed to a stressor in order to produce the target compound and the secondary signal; tested for magnitude of the secondary signal; and re-exposed to the stressor.

In one variation in which the genome of the fly population is genetically modified to include multiple unique target sequences configured to generate multiple unique target compounds, the genome can be further genetically-modified to include multiple reporter sequences 119. In this variation, the genome of the fly population can be genetically modified to include: a set of target sequences encoding for a set of target compounds; a set of promoter sequences, each promoter sequence coupled to a particular target sequence, in the set of target sequences, and associated with a particular stressor, in a set of stressors; and a set of reporter sequences 119, each reporter sequence 119 coupled to a particular target sequence, in the set of target sequences, and configured to express in response to expression of the particular target sequence.

### 4.3 Variation: Flightless Flies

In one variation, the fly population can be genetically modified to include a trait configured to inhibit flight of flies in the fly population. For example, a genome of the fly population can be genetically modified to include a curly-wing trait—encoding for curly wings—such that the resulting fly population includes curly-wing flies. In another example, a genome of the fly population can be genetically modified to encode for absence of wings, such that the resulting fly population includes wingless flies. By modifying the fly population to include flies with limited flight capabilities and/or incapable of flight (or “flightless flies”), loss of flies in the fly population—such as due to escape of flies (e.g., via flight) during feedings and/or transfer between cages—can be minimized, while eliminating a need to anesthetize flies (e.g., via injection of carbon dioxide into cages) during these feedings and/or cage transfers, thereby minimizing resources (e.g., cost, labor, materials) and reducing risk associated with anesthetizing flies.

### 4.4 Genetic Modification: Plasmid Generation and Insertion

In one implementation—as described in U.S. patent application Ser. No. 18/075,362, filed on 5 Dec. 2022, which is incorporated in its entirety by this reference—the fly population can be genetically modified via insertion of a plasmid (e.g., a pUAST plasmid, a pCaSpeR plasmid)—including the target sequence encoding for the target compound—into the genome of the fly population. The plasmid can also be configured to include a set of regulatory sequences, such as the promoter sequence and/or the leader sequence. In particular, the target sequence and these regulatory sequences can be cloned via restriction enzyme cloning into a plasmid including a multiple cloning site (or “MCS”). The target sequence corresponding to the target compound can be amplified via Polymerase Chain Reaction (or “PCR”) with the addition of restriction cut sites for a set (e.g., one or two) of the restriction enzymes added onto 5′ and 3′ amplification primers, thus enabling inserting of the target sequence into the vector at the MCS.

The resulting plasmid vector including the target sequence (e.g., a recombinant protein-coding sequence) can then be transformed into chemically competent bacterial cells for propagation. In particular, the plasmid vector can be transformed via ampicillin selection. Once the plasmid vector is propagated in the bacterial culture, the plasmid DNA can be extracted from the bacterial culture and purified. Successful insertion of the target sequence into the plasmid vector can be verified by sequencing upstream and downstream from the insertion site (i.e., the MCS).

Upon confirmation of insertion of the target sequence into the plasmid vector, the target sequence can be inserted into the genome of the fly population. Once the target sequence is integrated into the insect genome, the fly population can be propagated for production of the target compound.

In one example, a population of Drosophila can be genetically modified to produce a target compound via insertion of a recombinant pCaSpeR plasmid into Drosophila embryos. In this example, the plasmid can be configured to include: a target sequence encoding for a particular target compound; a set of regulatory sequences including a leader sequence upstream the target sequence, and a promoter sequence (e.g., an HSP70 promoter sequence) upstream of the leader sequence; and an ampicillin resistance gene configured to enable selection of transformants during propagation of the plasmid in bacterial cells; a set of P-element sites configured for insertion of the plasmid into Drosophila embryos; an MCS including a set of cut sites corresponding to a set of restriction enzymes (e.g., EcoRI, BgIII, Notl, Xhol, Kpnl, and Xba1); and a test sequence (e.g., corresponding to the white+ gene) configured to enable visual confirmation of propagation of the target sequence in the population of Drosophila.

In this example, the target sequence and regulatory sequences can be cloned via restriction enzyme cloning into the pCaSpeR plasmid. In particular, the target sequence and regulatory sequences can be amplified via PCR with the addition of restriction cut sites for a subset (e.g., one or two) of restriction enzymes, in the set of restriction enzymes, added onto 5′ and 3′ amplification primers, thereby enabling insertion of the target sequence into the pCaSpeR vector at the MCS via implementation of a T4 DNA ligase. Because the pCaSpeR vector includes the ampicillin resistance gene, the resulting pCaSpeR vector including the target sequence can then be transformed into chemically competent bacterial cells for propagation via ampicillin selection. After propagation of the pCaSpeR vector in the bacterial culture, the plasmid DNA can be extracted and purified. To confirm insertion of the target sequence into the pCaSpeR vector, the plasmid DNA can be sequenced upstream and downstream the MCS.

Once insertion of the target sequence into the pCaSpeR vector is confirmed, the target sequence can be inserted into the Drosophila genome via P-element insertion. A P-element transposon present in the plasmid vector at a P-element site, in the set of P-element sites, can be leveraged to integrate the exogenous target sequence into the insect genome in the presence of a transposase. In particular, the target sequence can be integrated into the Drosophila genome via germline transformation including microinjection of the plasmid DNA and a helper plasmid including transposase, into a recipient Drosophila embryo. Therefore, in this example, the plasmid DNA can be physically delivered to a posterior pole of the syncytial blastoderm at which precursors of the germ cells form. Upon cellularization of the embryo, the plasmid DNA—including the set of P-element sites—can be integrated into the genome of the germ cells via interactions between the transposase and P-elements at the set of P-element sites.

As described in the preceding example, in one implementation, the fly population can therefore be genetically-modified to produce the target compound via P-element based genome integration. However, the population of insects can be genetically-modified to produce the target compound via any other known genetic engineering technique. For example, in one implementation, the population of insects can be genetically-modified to produce the target compound via implementation of a site-specific integrase system. In particular, in this implementation, in this implementation, the population of insects can be genetically modified to produce the target compound via implementation of a recombinant pJFRC81 plasmid. Enzyme integrases can then be leveraged to integrate the exogenous target sequence into the insect genome via recombination between attachment sites (e.g., attB and attP) on the vector backbone and Drosophila genomes.

### 4.4.1 Example: Generation of Genetically-Modified Population of Drosophila

In one example, a population of Drosophila can be genetically modified to produce a target compound. In this example, the target sequence (i.e., coding sequence)—coding for the target compound (e.g., a target recombinant protein)—is cloned via restriction enzyme cloning into a pUAST plasmid including a multiple cloning site (or “MCS”). The target sequence is then amplified by PCR with the addition of restriction cut sites for one or two of the restriction enzymes added onto 5′ and 3′ amplification primers, thereby enabling insertion of the target sequence into the pUAST plasmid vector at the MCS via a T4 DNA ligase.

The resulting plasmid vector—including the target sequence (e.g., the recombinant protein sequence)—can then be transformed into chemically competent bacterial cells for propagation. In this example, this transformation can be performed under ampicillin selection due to the presence of an ampicillin resistance gene in the pUAST vector. After the plasmid vector is thus propagated in the bacterial culture, the plasmid DNA is extracted and purified. Insertion of the target sequence (e.g., the recombinant protein-coding sequence) into the pUAST vector can then be verified by sequencing upstream and downstream from the insertion site.

Upon confirmation of insertion into the plasmid (i.e., the pUAST plasmid vector), the target sequence can be stably inserted into the Drosophila genome. For example, the target sequence can be stably inserted into the Drosophila genome via a P-element transposon present in the plasmid vector. In particular, the pUAST vector includes P-element sites that—in the presence of a transposase—enable stable integration of exogenous DNA into the Drosophila genome. The target sequence can be inserted via germline transformation through microinjection of the modified plasmid DNA (i.e., the modified pUAST plasmid vector) and a helper plasmid—including transposase—into a recipient Drosophila embryo. In this example, the plasmid DNA (e.g., the modified pUAST plasmid vector) is delivered to the posterior pole of the syncytial blastoderm, at which precursors of the germ cells are formed. Thus, upon cellularization of the embryo, the plasmid DNA (e.g., the modified pUAST plasmid vector)—including P-elements—is integrated into the genome of the germ cells via activity of the transposase.

### 5. Rearing Schedule

The system 100 can be configured to rear a multitude of fly populations according to a rearing schedule defined for each fly population. In particular, the system 100 can include: a set of feeder modules 110, each feeder module 110, in the set of feeder modules 110, configured to cultivate a set of feeder populations of adult flies according to a feeder schedule defined for the particular feeder module 110; a set of incubator modules 120, each incubator module 120, in the set of incubator modules 120, configured to cultivate a set of incubator populations of eggs and larvae—derived from the set of feeder populations—according to an incubator schedule defined for the particular incubator module 120; and a set of repopulation modules 140, each repopulation module 140, in the set of repopulation modules 140, configured to cultivate a set of replacement populations of pre-adult stage flies (e.g., larvae, pupae)—derived from the set of incubator populations—according to a repopulation schedule defined for the particular repopulation module 140. The system 100 can therefore be configured to enable semi-continuous generation and collection of the target compound by enabling cyclical and continuous cultivation of fly populations according to the rearing schedule.

In one implementation, the system 100 can define a rearing schedule—configured to maximize production of the target compound—including: an egg-laying period, of a first target duration (e.g., 8 hours, 12 hours, 24 hours), during which a fly population—contained in a feeder cage 150 in the feeder module 110—consumes foodstuff applied to a surface of the feeder cage 150 and deposits eggs onto this surface; an incubation period, of a second target duration (e.g., 2 days, 3 days, 5 days), during which larvae—contained in an incubator cage 152 in the incubator module 120—emerge from eggs extracted from the feeder cage 150 and mature into fully-developed larvae, the second target duration exceeding the first target duration; a treatment period, of a third target duration (e.g., 4 hours, 8 hours, 12 hours, 24 hours), during which larvae—contained in the incubator cage 152 in a heat-shock chamber—are subjected to heat-shock according to a heat-shock protocol configured to induce production of the target compound in larvae; and a harvest period, succeeding the treatment period, during which larvae in the incubator cage 152 are harvested for extraction and collection of the target compound. In one example, the rearing schedule can include: an egg-laying period of approximately 24 hours; an incubation period—immediately succeeding the egg-laying period—of approximately 65 hours; a treatment period—immediately succeeding the incubation period—of a duration between approximately 4 hours and 8 hours; and a harvest period—immediately succeeding the first treatment period—during which larvae are harvested from the incubator cage 152 for extraction and collection of the target compound. In this example, the rearing schedule can define a series of egg-laying periods for a single feeder population, such that batches of fly eggs can be regularly collected after each egg-laying period and transferred to an incubator cage 152 for a subsequent incubation period. Therefore, the system can continuously repeat this process to generate and collect multiple batches of the target compound from this single feeder population over a life cycle of this feeder population.

Additionally, in the preceding implementation, in order to enable continuous generation of the target compound, the rearing schedule can include: a growth period, succeeding the incubation period in replacement of the treatment period and the harvest period at a fixed frequency (e.g., once per week, once every two weeks), during which larvae—contained in a pupae cage in a replenishment module—mature into pupae from which adult flies emerge. These adult flies—defining a next generation of the fly population—can then replace the fly population contained in the feeder cage 150, in the feeder module 110.

### 6. Modular Rearing System

The system 100 can be distributed throughout a facility configured to enable execution of Blocks of the method S100. In particular, the system 100 can include: a set of rearing modules distributed throughout the facility and configured to cultivate fly populations (e.g., adult flies and/or fly larvae) to promote generation of a target compound in these fly populations; a set of environmental controls 180 configured to regulate ambient conditions—such as air temperature, humidity, pressure, and/or flow—at each of these rearing modules to a target set of ambient conditions defined for each particular module; and a distributed sensor suite configured to regularly collect ambient sensor data from various modules.

For example, the system 100 can include: a feeder module 110 assigned to a first area (e.g., a feeder area) of the facility and defining a first array of feeder cage slots assigned to feeder cages 150 containing adult flies configured to generate a first target compound type; an incubator module 120 assigned to a second area (e.g., an incubator area) of the facility and defining an array of incubator cage slots configured to receive incubator cages 152; a repopulation module 140 assigned to a third area (e.g., a repopulation area) of the facility and defining a third array of repopulation cage slots configured to receive repopulation cages 154; a suite of sensors distributed between the feeder module 110, the incubator module 120, and the repopulation module 140 and configured to record temperature and humidity data at each module; and a set of environmental controls 180 configured to regulate temperature and humidity at each module.

### 6.1 Rearing Modules

As described above, the system 100 can include a set of rearing modules (or “modules”) of various types, such as including adult-fly rearing modules (hereinafter “feeder modules 110”), egg and larvae rearing modules (hereinafter “incubator” modules) and feeder repopulation modules 140 (hereinafter “repopulation modules 140”).

Each module of the system 100 is configured to house a group of fly populations—including fully-matured adult fly populations (hereinafter “feeder populations”), egg and larvae populations (hereinafter “incubator populations”), and later-stage larvae and pupae populations (hereinafter “replacement populations”)—throughout a segment of the fly growth cycle.

In particular, in one implementation, each module can include a carriage or frame defining an array of cage slots (e.g., arranged in one or more columns); and each cage slot can receive and retain a cage corresponding to the module, such as a feeder cage 150 containing an adult fly population, an incubation cage containing a larvae population, and/or a regeneration cage containing a maturing fly population. Each module can define a standard size (e.g., 2 feet in width by 2 feet in length by 5 feet in height; 1 meter in width by 1 meter in length by 2 meters in height; 10 feet in width by 10 feet in length by 8 feet in height) and a standard spacing (e.g., 4 inches, 8 inches, 12 inches)—equivalent and/or marginally exceeding a height of the cage—between adjacent cage slots, such that each cage slot in the module can simultaneously be loaded with a cage. Each module can therefore be configured to maximize a number of cages loaded within the module while minimizing dead space and/or gaps between cages contained in cage slots in the module.

Additionally, in one implementation, each module can be configured for transportation within a space or facility. For example, each module can include a set of wheels coupled to a bottom of the carriage. Additionally and/or alternatively, in another example, each module can include a set of engagement features integrated within and/or extending outward from the carriage and configured to enable maneuvering of the carriage, such as by a robotic mover or a human user.

Additionally or alternatively, in one implementation, the facility can include: a first enclosed environment (e.g., a room, a chamber, an enclosed region) arranged in a first region of the facility and assigned to feeder modules; a second enclosed environment arranged in a second region of the facility and assigned to incubator modules; a third enclosed environment arranged in a third region of the facility and assigned to treatment modules; and/or a fourth enclosed environment arranged in a fourth region of the facility and assigned to repopulation modules.

In this implementation, the system can include a first set of feeder modules arranged within the first enclosed environment. For example, the system can include a first feeder module—such as spanning a length and/or width of the first enclosed environment—defining an array of cage slots transiently loaded with an array of feeder cages. Alternatively, in another example, the system can include twenty feeder modules—such as arranged in a row(s) across the enclosed environment—each defining an array of cage slots. In this example, each of the twenty feeder modules can be configured for individual transportation within the facility.

In the preceding implementation, the facility can further include additional enclosed environments assigned to various modules, such as including: a first enclosed environment arranged in a first region of the facility and assigned to feeder modules housing fly populations modified to produce a first target compound; and a second enclosed environment arranged in a second region of the facility and assigned to feeder modules housing fly populations modified to produce a second target compound differing from the first target compound. In this example, the facility can therefore be configured to separate fly populations of differing genomes, thereby reducing risk of cross-contamination and enabling independent environmental regulation—such as via the set of environmental controls, as described below—of flies in these populations.

### 6.2 Environmental Controls

In one implementation, the system 100 can include a set of environmental controls 180—configured to regulate ambient conditions—such as air temperature, humidity, pressure, and/or flow—at a particular module (e.g., within an enclosed environment housing the particular module) according to a target set of ambient conditions defined for the particular module. Additionally, the set of environmental controls 180 can be configured to regulate these ambient conditions throughout the enclosed environment—according to the target set of ambient conditions—to achieve substantially uniform ambient conditions at each cage, at each position, within the module.

In one implementation, each module can include a module housing or covering (e.g., a plastic cover, a rigid housing) configured to encapsulate the array of cage slots to locate feeder cages 150—loaded in the array of cage slots—within an enclosed environment defined by the module covering. For example, the system 100 can include: a feeder module 110 defining an array of 20 cage slots arranged in a vertical stack and loaded with a set of feeder cages 150; a temperature control coupled to the feeder module 110 and configured to regulate temperature within the feeder module 110 toward a target temperature defined for the feeder module 110; and a humidity control coupled to the feeder module 110 and configured to regulate humidity within the feeder module 110 toward a target humidity defined for the feeder module 110. The temperature control can therefore be configured to regulate temperature within the feeder module 110—according to the target temperature—and achieve a substantially uniform temperature profile throughout the feeder module 110, such as from a first cage loaded in the first cage slot at a top of the vertical stack to a tenth cage loaded in the tenth cage slot at a bottom of the vertical stack.

In one example, a feeder module 110 can include: a carriage defining an array of cage slots—loaded with a set of feeder cages 150 containing feeder populations (e.g., adult fly populations)—arranged in a vertical stack; and a plastic covering arranged about the carriage and defining an enclosed environment—isolated from an external environment surrounding the plastic covering—containing the set of feeder cages 150. In this example, the system 100 can include: an internal fan—arranged within the enclosed environment, proximal a bottom of the carriage, and coupled to an external power source—configured to blow air upward within the internal environment to promote uniformity of temperature and humidity throughout the internal environment; and an external fan fluidly coupled to the internal environment—such as via an air inlet arranged on the plastic covering—and configured to inject dry air into the internal environment to regulate temperature and humidity within the internal environment toward target temperature and humidity values defined for the feeder module 110.

Additionally or alternatively, in one implementation, each module can be located in a particular region of the facility—such as an enclosed room—configured to house the module according to the target set of conditions defined for this particular module. For example, the system 100 can include: a set of feeder modules (e.g., one or more feeder modules) arranged within a first room of a facility; a set of incubator modules arranged within a second room of the facility; and a set of repopulation modules arranged within a third room of the facility. In this example, the system 100 can include: a first temperature control installed within the first room and configured to regulate temperature within the first room to regulate temperature of the set of feeder modules to a first target temperature defined for a feeder module; a second temperature control installed within the second room and configured to regulate temperature of the set of incubator modules to a second target temperature defined for an incubator module; and a third temperature control installed within the third room and configured to regulate temperature of the set of repopulation modules to a third target temperature defined for an repopulation module. The system 100 can similarly include additional environmental controls (e.g., pressure controls) arranged within each room of the facility to independently regulate environmental conditions within each of these rooms based on a type of module housed within the room.

### 6.3 Sensor Suite

In one implementation, the system 100 can include a fixed sensor suite configured to regularly collect ambient sensor data from various modules, such as once per hour or once per second, and return this data to the computer system.

For example, the system 100 can include a suite of sensors including: a set of humidity sensors configured to record relative humidity at modules; a set of temperature sensors configured to record temperature at modules; and a pressure sensor configured to record air pressure within cages (e.g., feeder cages 150 and/or incubator cages 152) at modules. The suite of sensors can thus record ambient sensor data (e.g., temperature, pressure, humidity) at a fixed frequency; and return these data to the controller 160 and/or computer system via a wired or wireless connection. Based on these data, the controller 160 and/or computer system can then selectively actuate the set of environmental controls 180 to regulate humidity, temperature, and/or pressure within these modules.

In one example, the system 100 includes: a first set of sensors configured to capture environmental data representing conditions at a set of feeder modules (e.g., one or more feeder modules), such as installed within a region (e.g., an enclosed room, a covered area) of the facility assigned to feeder modules; a second set of sensors configured to capture environmental data representing conditions at a set of incubator modules, such as installed within a region of the facility assigned to incubator modules; a third set of sensors configured to capture environmental data representing conditions at a set of treatment modules, such as installed within a region (e.g., a chamber, a thermally-isolated room) of the facility assigned to treatment modules; and/or a fourth set of sensors configured to capture environmental data representing conditions at a set of repopulation modules, such as installed within a region of the facility assigned to repopulation modules.

### 7. Reconfigurable Cages

The system 100 can include a set of reconfigurable cages including: a set of feeder cages 150—each feeder cage 150 containing a feeder population of adult flies—assigned to the feeder module 110; a set of incubator cages 152—each incubator cage 152 containing an incubator population of fly eggs and/or fly larvae—assigned to the incubator module 120; and a set of repopulation cages 154—each repopulation cage 154 containing a replacement population of fly larvae, fly pupae, and/or adult flies—assigned to the repopulation module 140. Each reconfigurable cage (hereinafter “cage”) can be configured to: house a fly population in a particular stage of the fly life cycle (e.g., adult flies, eggs, larvae, or pupae); and prevent escape of flies in the fly population from the cage.

Generally, each cage can include a tray 158 and a food container 156 removably coupled to the tray 158. The tray 158 can define: a base surface; tray 158 walls extending from a perimeter of the base surface and defining a first height; and a tray 158 rim—extending from the tray 158 walls opposite the base surface—defining a first set of coupling features. The food container 156 is removably coupled to the tray 158 and defines: a food surface; container walls extending from a perimeter of the food surface and defining a second height; and a container rim—extending from the container walls opposite the food surface—defining a second set of coupling features configured to transiently couple to the first set of coupling features to form a cage. The food container 156 further includes a layer of foodstuff (hereinafter a “food layer”): arranged on the food surface; configured for consumption by fly populations at each stage of the fly life cycle to sustain these fly populations; and configured to receive and retain eggs laid by adult flies in a feeder population contained in a feeder cage 150.

In one implementation, the food container 156 can be configured to: couple to a first tray 158 to form a feeder cage 150—containing a feeder population—assigned to the feeder module 110 during an egg-laying period of a target duration; and, in response to expiration of the target duration, couple to a second tray 158—in replacement of the first tray 158—to form an incubator cage 152 containing an incubator population and assigned to the incubator module 120 during an incubation period succeeding the egg-laying period. The food container 156—including the food layer—can therefore: supply food to the feeder population and collect eggs deposited by the feeder population, within the food layer, during the egg-laying period; and retain and locate these eggs within the incubator module 120 and supply food to larvae—hatched from the eggs—in the incubator population during the incubation period, such as without necessitating transfer of eggs into a new container or loading of additional foodstuff into the incubator cage 152. Further, in response to expiration of the target duration, a new food container 156—including a fresh food layer—can be coupled to the first tray 158, in replacement of the previous food container 156, to reassemble the feeder cage 150 in preparation for a subsequent egg-laying period for the feeder population.

Each cage—including feeder, incubator, and repopulation cages 154—can define a standard cage size corresponding to a standard slot size of each cage slot in each module. In particular, each tray 158 can define a standard tray 158 size and each food container 156 can define a standard container size, such that each food container 156 is configured to couple with each tray 158 across different modules.

In one implementation, each cage can define a particular standard cage size, such that the cage is configured to: house and cultivate a fly population of a target population size; and fit within a target footprint—such as defined by the cage slot—in order to maximize a quantity of feeder cages 150 stored within the feeder module 110. In this implementation, each cage can define a target population size—such as for the feeder and/or incubator populations contained within the cage—less than a maximum population size and exceeding a minimum population size. In particular, by maintaining a population size of the feeder population below the maximum population size, the system 100 can: limit competition between adult flies in the feeder population for space and food; minimize an amount of food required to feed the feeder population; and minimize sensitivity of downstream outputs—such as production of the target compound—to complications (e.g., fly death, disease spread, flies escaping, propagation of undesirable fly traits, cross-breeding) with this particular fly population and therefore contain complications or accidents to this singular fly population without experiencing major delays or decreases in production of the target compound. Further, by maintaining the population size of the feeder population above the minimum population size, the system 100 can maximize production of the target compound per area (e.g., defined by the cage), such as by maximizing a quantity of eggs laid by adult flies on the food layer in the food container 156.

Each cage can therefore define a standard size—such as defining outer dimensions and/or the internal volume—corresponding to the target population size and the target footprint. In particular, each cage can define a standard size that is configured to: minimize a footprint of the cage—within the modules and/or within a facility containing these modules—in order to maximize a quantity of cages located within these modules and/or within the facility; and maximize a population size of the fly population contained within the feeder cage 150—and therefore maximize an amount of the target compound generated downstream—while promoting health and propagation of flies within the feeder cage 150.

### 7.1 Feeder Cage: Inverted Configuration

Each feeder module 110 can be configured to receive and retain a set of feeder cages 150 (e.g., arranged in a vertical stack), each feeder cage 150, in the set of feeder cages 150, containing a feeder population of adult flies. Each feeder cage 150 can be configured to: house a discrete feeder population within an interior volume defined by the feeder cage 150; inhibit escape of adult flies, in the feeder population, from within the feeder cage 150; enable regular feeding of adult flies in the feeder population, such as according to a feeding schedule defined for the feeder module 110; and collect eggs deposited by adult flies, in the feeder population, for propagating within the incubator module 120.

In particular, each feeder module 110 can be configured to receive and retain a set of feeder cages 150—arranged in an inverted configuration, such that the food surface of the food container 156 is arranged vertically above the base surface of the tray 158—within an array of cage slots defined by the feeder module 110. For example, each feeder cage 150—in the inverted configuration—can define: an upper cage region defined by the food container 156 and including the food layer; and a lower cage region defined by the tray 158. Adult flies in the feeder population—which generally seek moist, decaying organic material for deposition of eggs—may thus fly or crawl from the lower cage region to the upper cage region to concurrently consume foodstuff and deposit eggs in the food layer.

As described above, each feeder module 110 can define a feeder schedule defining instructions for rearing a set of feeder populations located in the feeder module 110. In particular, the feeder schedule can specify: a quantity of feed cycles for feeder populations within the feeder module 110; and a fixed duration (e.g., 24 hours) of each of these feed cycles. Upon completion of a particular feed cycle, the food container 156 can be collected and located in the incubator module 120 for incubation of eggs deposited by the feeder population in the food layer and replaced by a new food container 156 for the subsequent feed cycle. The food container 156 coupled to the tray 158 of the feeder cage 150 can therefore be regularly (e.g., according to the feeder schedule) retrieved from the feeder cage 150 and replaced by a new food container 156 including a fresh food layer, such as at a frequency of once every 24 hours.

By thus locating the feeder cage 150 in the inverted configuration in the feeder module 110, the system 100 can enable removal of the food container 156—containing eggs deposited by the feeder population on the food layer—by leveraging gravity to confine flies to the lower region (i.e., the tray 158) of the feeder cage 150 prior to and during removal of the food container 156. For example, a volume of carbon dioxide can be injected into the cage in order to transiently incapacitate (e.g., paralyze, anesthetize) adult flies, in the feeder population, and thus locate the feeder population—in a dormant state—on the base surface of the tray 158 via gravitational forces. The food container 156—containing eggs deposited in the food layer and emptied of any adult flies—can then be removed and replaced with a new food container 156 prior to flies in the feeder population transitioning from the dormant state to a live state. Therefore, each feeder cage 150 can be configured to prevent escape of adult flies from the feeder population during swapping of the food container 156 and enable rapid collection of the food container 156—containing eggs deposited by the feeder population—with minimal loss in egg yield.

In one implementation, in order to enable maintaining of the feeder cage 150 in the inverted configuration, the food layer can be configured to exhibit a texture that is substantially firm in order to withstand mechanical action, such that foodstuff in this layer: sticks to the food surface of the food container 156 (i.e., the upper surface of the feeder cage 150) in the inverted configuration and thus does not fall downward onto the base surface of the tray 158; and retains eggs deposited by the feeder population within the food layer against a gravitation pull exerted on these eggs.

### 7.1.1 Fly Selection: Inverted Configuration

Additionally, in one implementation, the feeder cage 150 can define a gap—extending vertically between the base surface of the tray 158 and the food surface of the food container 156—of a target height, such that adult flies in the feeder population contained in the feeder cage 150 must traverse this gap in order to consume foodstuff from the food layer applied across the food surface of the food container 156. Therefore, by arranging the food layer vertically above and offset the base surface of the tray 158, the feeder cage 150 requires adult flies in the feeder population to exert at least a threshold energy to travel from the bottom surface and upward—across this gap—toward the food layer, such as by flying and/or crawling up perimeter walls of the food cage.

Therefore, by enabling consumption of food by adult flies capable of traversing this gap between the bottom surface and the food layer, the feeder cage 150 can promote egg-laying by these relatively healthy flies and thus promote selection of healthy flies while selecting against adult flies exhibiting poorer health. The feeder cage 150 can therefore be configured to: maximize collection of eggs laid by healthy adult flies in the feeder population, thereby maximizing a probability of yielding healthy larvae and/or adult flies (e.g., in a future feeder population) from these eggs; and minimize collection of eggs laid by unhealthy or weaker adult flies, thereby minimizing a probability of yielding unhealthy larvae and/or adult flies that may produce lower quantities of the target compound and/or exhibit a lower likelihood of survival and/or propagation.

### 7.1.2 Fly Selection: Cage Ramps+Inverted Configuration

In one variation, the feeder cage 150 can include a set of ramps—extending from the bottom surface of the feeder cage 150 toward the food layer arranged across the food surface of the food container 156—configured to enable flies, in the population of adult flies contained within the feeder cage 150, to access the layer of foodstuff for consumption via traversing (e.g., crawling, climbing) the set of ramps, such as without flying.

In particular, in this variation, the population of adult flies can be genetically modified to include a trait (e.g., a curly-wing trait) configured to inhibit flight in flies including the trait, thereby inhibiting escape of flies from the feeder cage 150—such as during replacement of the food container 156—and preventing cross-contamination between populations of adult flies in other feeder cages 150. The tray 158 of the feeder cage 150 can therefore include a set of ramps configured to enable these “flightless” flies—or flies genetically modified to include a trait (e.g., a curly-wing trait) inhibiting flight—to access the food layer arranged above bottom surface of the tray 158 in the inverted configuration, each ramp, in the set of ramps: defining a lower end coupled to the bottom surface of the tray 158; defining an upper end—vertically offset the lower end by approximately a height of the feeder cage 150—configured to contact and/or seat within a threshold distance of a food layer of a food container 156 coupled to the tray 158 in the inverted configuration; and extending at a target angle (e.g., 30 degrees, 45 degrees, 90 degrees)—from the bottom surface of the tray 158—configured to enable traversal of relatively healthy flies from the first end to the second end while inhibiting traversal of relatively unhealthy flies from the first end to the second end. The feeder cage 150 can therefore be configured to: promote selection of relatively healthy flies—such as characterized by fitness level, strength, activity level, lifespan, quantities of eggs produced, viability of eggs produced, disease(s), etc.—while selecting against relatively unhealthy adult flies, as less healthy and/or unhealthy flies may experience difficulty (and/or may not attempt) climbing upward from the lower end toward the upper end of the ramp; and thus promote generation of relatively healthy and/or viable offspring descending from adult flies in the feeder cage 150, thereby increasing production of the target compound.

Furthermore, the feeder cage 150 can be configured to house a fly population of a target size—such as including a target quantity of adult flies—proportional an internal surface area of the tray 158. Therefore, by including the set of ramps, the cage can define a greater internal surface area—configured to receive flies in the (flightless) fly population—thereby increasing a fly capacity of the cage without increasing an internal volume of the cage. Each feeder cage 150 can therefore be configured to include a set of ramps—such as rigidly and/or flexibly coupled to the bottom surface of the tray 158—in order to: enable consumption of food in the food layer by flightless flies in the fly population; maximize internal surface area of the feeder cage 150 formed of a food container 156 transiently coupled to the tray 158; maximize a fly capacity—or quantity of flies, in the fly population, contained in the feeder cage 150; minimize a volume of the feeder cage 150—and therefore minimize a volume of a feeder module 110 transiently loaded with an array of feeder cages 150—required to house the fly population at the fly capacity; and/or maximize an amount of the target compound generated per volume of the feeder cage 150.

### 7.2 Incubator Cage: Upright Configuration

Each incubator module 120 can be configured to receive and retain a set of incubator cages 152 (e.g., arranged in a vertical stack), each incubator cage 152, in the set of incubator cages 152, containing an incubator population of fly eggs and/or larvae. Each incubator cage 152 can be configured to: house a discrete incubator population within an interior volume defined by the incubator cage 152; contain the incubator population within the interior volume; enable consumption of foodstuff—in the food layer in the container—by the incubator population; and enable harvesting of the incubator population from the incubator cage 152, such as according to an incubation schedule defined for the incubator module 120.

In particular, each incubator module 120 can be configured to receive and retain a set of incubator cages 152—arranged in an upright configuration, such that the food surface of the food container 156 is arranged vertically below the base surface of the tray 158—within an array of cage slots defined by the incubator module 120. For example, each incubator cage 152—in the upright configuration—can define: a lower cage region defined by the food container 156 and including the food layer; and an upper cage region defined by the tray 158. By thus locating the incubator cage 152 in the upright configuration within the incubator module 120, the incubator cage 152 can be configured to locate the incubator population—incapable of flying and/or crawling up walls of the incubator cage 152—on or within the food layer, in the lower region of the incubator, and thereby enable consumption of foodstuff by the incubator population.

In one implementation, the incubator cage 152 can be configured to contain a target amount of foodstuff at a start of an incubation cycle, such that the food layer defines a target height—extending upward from the food surface—at the start of the incubation cycle. In particular, as described above, each incubator module 120 can define an incubation schedule defining instructions for rearing a set of incubator populations located in the incubator module 120. In particular, the incubator schedule can specify a target duration of an incubation cycle for incubator populations within a particular incubator module 120. Upon completion of the incubator schedule, the food container 156—including any remaining foodstuff and the incubator population—can be collected and rinsed with a volume of water, which can then be filtered to extract the incubator population from the volume of water and any water-soluble material (e.g., digested foodstuff). Therefore, because fly larvae externally digest food (e.g., by liquefying food external the larvae body)—and thereby convert non water-soluble foodstuff to water-soluble foodstuff—the incubator cage 152 can be configured to include this target amount of foodstuff, such that the incubator population may convert this foodstuff to water-soluble material prior to completion of the incubation cycle.

### 7.3 Repopulation Cage

Each repopulation module 140 can be configured to receive and retain a set of repopulation cages 154 (e.g., arranged in a vertical stack), each repopulation cage 154, in the set of repopulation cages 154, containing a replacement population of developing flies (e.g., including larvae, pupae, and/or adult flies). Each repopulation cage 154 can be configured to: house a discrete replacement population within an interior volume defined by the repopulation cage 154; contain the replacement population within the interior volume; enable consumption of foodstuff—in the food layer in the container—by the repopulation population; and enable transfer of the final replacement population of adult flies to a feeder cage 150 for locating in the feeder module 110.

### 8. Target Compound Generation Cycle

Generally, a first fly population can be genetically modified to produce amounts of a target compound. The first fly population can be located in a feeder cage 150 arranged within a feeder module 110 defining an array of cage slots configured to transiently receive and retain feeder cages 150. Then, at a fixed or variable frequency (e.g., once per day), a population of eggs can be collected from the feeder cage 150, such as by replacing the food container 156—loaded with the population of eggs deposited by the first fly population—with a new food container 156. The population of eggs contained in the food container 156 can then be transferred to an incubator cage 152—formed of the food container 156 coupled to a new tray 158—and located within an incubator module 120 configured to promote transformation of fly eggs into fly larvae and defining an array of cage slots configured to transiently receive and retain incubator cages 152. Then, in response to expiration of a target duration defined for incubation, the incubator cage 152—now containing a population of larvae derived from the population of eggs—can be transferred from the incubator module 120 to a treatment module 130 (e.g., a heat-shock chamber) configured to promote generation of the target compound. The population of larvae can then be harvested from the second cage for extraction of amounts of the target compound from these larvae.

### 8.1 Feeding Period

Block S110 of the method S100 recites: during a first feeding period of a first fixed duration, subjecting a first set of cages—occupied by a set of fly populations and arranged within a feeder module 110 in an inverted configuration—to a first set of environmental conditions (or “feeder conditions”) configured to promote deposition of eggs in the first set of cages by adult flies in the set of fly populations.

Generally, during a feeding period of a fixed duration (e.g., 12 hours, 24 hours, 36 hours), a feeder cage 150—including a first population of adult flies genetically-modified to generate amounts of a first target compound—can be exposed to a first set of environmental conditions (e.g., humidity, temperature) configured to promote deposition of eggs by adult flies within the first cage.

In one implementation, the feeder cage 150 can be arranged in a feeder module 110 transiently loaded with a set of feeder cages 150, including the feeder cage 150, and the feeder module 110 (e.g., including the set of feeder cages 150) can be subjected to a first set of environmental conditions—including temperatures within a threshold deviation of a target feeding temperature and pressures within a threshold deviation of a target feeding pressure—configured to maximize egg-laying activity in the fly population and therefore maximize a quantity of eggs deposited by adult flies, in the fly population, within the feeder cage 150. For example, throughout the fixed duration of the feeding period, the feeder cage 150 can be exposed to: a target humidity of approximately (e.g., within five percent) 70 percent relative humidity; and a target temperature of approximately (e.g., within five percent) 25 degrees Celsius.

In one implementation, at a start of a first feeding period of the fixed duration, a first food container 156, in a set of food containers 156, loaded with a (fresh) food layer can be coupled to a first tray 158—populated by a first fly population of adult flies genetically-modified to produce amounts of a first target compound—to form a first feeder cage 150 (i.e., a cage transiently arranged in the feeder module 110) arranged in an inverted configuration, such that the food layer seats over and faces a bottom surface of the first tray 158.

The first feeder cage 150 can then be: located in a feeder module 110, such as including a first set of feeder cages 150—each feeder cage 150 populated by a fly population configured to produce amounts of a target compound in a set of target compounds—arranged in the inverted configuration within the feeder module 110; and, throughout the fixed duration of the first feeding period, subjected to a first set of environmental conditions—such as including a first air temperature and/or a first relative humidity—configured to promote consumption of foodstuff in the food layer and/or laying of eggs by adult flies in the first fly population.

Then, in response to expiration of the first fixed duration, the first feeder cage 150 can be removed from the feeder module 110 for replacement of the first food container 156—now loaded with a population of eggs deposited by the fly population during the first feeding period—with a second food container 156 loaded with a (fresh) food layer. In particular, in response to expiration of the first fixed duration, the first food container 156 can be decoupled from the first tray 158 for collection of the population of eggs deposited onto the first food container 156 by the fly population during the first feeding period. As described below, the first food container 156—including the population of eggs and decoupled from the first tray 158—can then be transferred to an incubation module configured to promote transformation of fly eggs into fly larvae.

Then, during a second feeding period succeeding (e.g., immediately succeeding) the first feeding period and of the first fixed duration, a second food container 156—including a second food layer (e.g., foodstuff)—can be coupled to the first tray 158 to re-form the first feeder cage 150 arranged in the inverted configuration, such that the second food layer seats over and faces the bottom surface of the first tray 158. During the second feeding period, the first feeder cage 150 can then be: located in the feeder module 110; and subjected to the first set of environmental conditions—configured to promote consumption of foodstuff in the second food layer and/or laying of eggs by adult flies in the first fly population—throughout the first fixed duration. Then, in response to expiration of the first fixed duration, the second food container 156—now including a second population of fly eggs—can be collected and replaced with a third food container 156 including a third food layer.

A series of feeding cycles—each feeding cycle of the first fixed duration—can therefore be executed to: replace a food layer—including an amount of foodstuff—supplied to the fly population at a fixed feeding frequency (e.g., corresponding to the first fixed duration); promote continuous and/or semi-continuous deposition of fly eggs by the first fly population; and regularly collect populations of fly eggs deposited by flies in the first fly population onto food containers 156 transiently coupled to the first tray 158 occupied by the first fly population. In one example, a first feeding cycle can be executed during a first 24-hour time period to generate a first population of eggs deposited by the first fly population; a second feeding cycle can be executed during a second 24-hour time period immediately succeeding the first feeding cycle—such that the first and second feeding cycle can be executed within a 48-hour time period—to generate a second population of eggs deposited by the first fly population; a third feeding cycle can be executed during a third 24-hour time period immediately succeeding the second feeding cycle to generate a third population of eggs deposited by the first fly population; etc.

### 8.2 Incubation Period

Block S120 of the method S100 recites: during a first incubation period—succeeding the first feeding period—of a second fixed duration, subjecting a second set of cages—occupied by a population of fly eggs and/or fly larvae (e.g., emerged from the population of fly eggs) and arranged within an incubation module in an upright configuration—to a second set of environmental conditions (or “incubator conditions”) configured to promote transformation of the population of fly eggs into a first population of fly larvae.

Generally, during an incubation period of a fixed duration (e.g., 24 hours, 36 hours, 96 hours, hours), an incubator cage 152—initially including a first population of fly eggs deposited by a first population of adult flies genetically-modified to generate amounts of a first target compound—can be exposed to a second set of environmental conditions (e.g., humidity, temperature) configured to promote transformation of the first population of fly eggs into a first population of fly larvae.

In one implementation, the incubator cage 152 can be arranged in an incubator module 120 transiently loaded with a set of incubator cages 152, including the incubator cage 152, and the incubator module 120 (e.g., including the set of incubator cages 152) can be subjected to a second set of environmental conditions—including a temperature ramp (e.g., an increase in temperature) from a first incubation temperature to a second incubation temperature exceeding the first incubation temperature and/or an air pressure within a threshold deviation of a target incubation pressure—configured to promote emerging of larvae from fly eggs and growth of these larvae within the incubator cage 152. For example, throughout the fixed duration of the incubation period, the incubator cage 152 can be exposed to: humidities within a target humidity range of approximately (e.g., within five percent) 70 percent relative humidity to 85 percent relative humidity; an initial incubator temperature of approximately 25 degrees Celsius at an initial time of the incubation period; a final incubator temperature of approximately 27 degrees Celsius at a final time of the incubation period; and a temperature ramp of approximately 2 degrees Celsius—from the initial incubator temperature at the initial time to the final incubator temperature at the final time—during the incubation period.

In one implementation, a food container 156 collected from the feeder module 110 and including a population of eggs can be coupled to a tray 158—excluding any fly populations—to form an incubator cage 152, including the population of eggs, in a set of incubator cages 152 arranged in the upright configuration in the incubator module 120.

In particular, as described above, during a first feeding period, a first food container 156 can be coupled to a first tray 158—containing a first fly population of adult flies genetically modified to produce amounts of a first target compound—to form a first feeder cage 150 arranged in an inverted configuration in the feeder module 110. Then, in response to expiration of a first fixed duration (e.g., 24 hours) of the first feeding period, the first food container 156 can be decoupled from the first tray 158 for collection of fly eggs deposited into the first food container 156 and replacement of the first food container 156 with a second food container 156 in preparation for a subsequent feeding cycle.

Then, during a first incubation period of a second fixed duration, the first food container 156—including the population of eggs deposited by the first fly population—can be coupled to a second tray 158 to form a first incubator cage 152, in a set of incubator cages 152, arranged in an upright configuration, such that the population of eggs seats below and facing an interior volume of the second tray 158. Then, during the first incubation period, the first incubator cage 152 can be: located in an incubator module 120, such as including a first set of incubator cages 152—each incubator cage 152 initially populated by a population of fly eggs derived from a fly population in the feeder module 110—arranged in the upright configuration within the incubator module 120; and, throughout the second fixed duration of the first incubation period, subjected to a second set of environmental conditions—such as including target air temperatures and/or target air pressures within the first incubator cage 152—configured to promote emerging of fly larvae from fly eggs and growth of these fly larvae within the set of incubator cages 152 throughout the incubation period.

Then, in response to expiration of the second fixed duration, the first incubator cage 152—containing a population of fly larvae emerged from the population of eggs and grown to a target size and/or a target larval stage—can be removed from the incubator module 120 in preparation for a treatment period, as described below.

### 8.3 Treatment Period

Block S130 of the method S100 recites: during a first treatment period succeeding the first incubation period and of a third fixed duration, subjecting a set of larvae cages—occupied by populations of fly larvae and removed from the incubator module 120—to a third set of environmental conditions (or “treatment conditions”) configured to promote generation of the first target compound in fly larvae contained in the set of larvae cages.

Generally, during a treatment period of a third fixed duration (e.g., one hour, four hours, eight hours, 24 hours), an incubator cage 152—including a population of fly larvae descending from a population of adult flies genetically-modified to generate amounts of a first target compound—can be exposed to a third set of environmental conditions (e.g., temperature, humidity, light) configured to promote generation of the first target compound in larvae in the population of fly larvae within the incubator cage 152. In particular, the population of fly larvae—grown from a population of eggs deposited by the population of adult flies—can be configured to generate the first target compound responsive to expression of a target sequence encoded in a genome of the population of fly larvae and the population of adult flies. Therefore, during the treatment period, the incubator cage 152 can be exposed to a third set of environmental conditions configured to induce expression of the target sequence in fly larvae, in the population of fly larvae, to generate amounts of the target compound within these fly larvae.

In one implementation, the incubator cage 152 can be: removed from the incubator module 120 in response to expiration of the second fixed duration of the incubation period; and arranged in a treatment module 130—transiently loaded with a set of incubator cages 152—during a treatment period of a third fixed duration. During the treatment period, the treatment module 130 (e.g., including the set of incubator cages 152) can be subjected to a third set of environmental conditions—such as including exposure to a particular stressor (e.g., heat-shock, cold-shock, nutrient deprivation, dehydration, UV exposure), a target temperature range and/or ramp, a target pressure ramp, etc.—configured to promote generation of the first target compound in fly larvae in the incubator cage 152. In one example, during a treatment period of the third fixed duration (e.g., four hours, eight hours), the incubator cage 152 can be exposed to: a heat shock stressor—including a treatment temperature within a treatment temperature range of approximately (e.g., within five percent, within ten percent) 37 degrees Celsius to approximately 41 degrees Celsius; and a target treatment humidity of approximately 80 percent relative humidity.

In particular, in this implementation, the incubator cage 152—arranged in the treatment module 130 in the upright configuration—can be exposed to a particular stressor configured to trigger expression of the target sequence and therefore generation of the first target compound. During the treatment period, the incubator cage 152 can therefore be exposed to a stressor selected based on a promoter sequence upstream the target sequence in a genome of fly larvae in the larvae population.

For example, a first incubator cage 152, in a first set of incubator cages 152, arranged in the treatment module 130 during a first treatment period and containing a first larvae population—defining a genome including a first target sequence encoding for a first target compound and a first promoter sequence upstream the first target sequence and associated with a heat-shock stressor—can be exposed to the heat-shock stressor (e.g., a temperature range from 37 degrees Celsius to 41 degrees Celsius) to trigger generation of the first target compound during the first treatment period. Alternatively, in this example, a second incubator cage 152, in a second set of incubator cages 152, arranged in the treatment module 130 during a second treatment period and containing a second larvae population—defining a genome including the first target sequence encoding for the first target compound and a second promoter sequence upstream the first target sequence and associated with a UV-light stressor—can be exposed to the UV-light stressor (e.g., UV light of a target intensity) to trigger generation of the first target compound during the second treatment period.

Then, in response to expiration of the third fixed duration of the treatment period, the first incubator cage 152—containing the first population of fly larvae—can be removed from the treatment module 130 in preparation for harvesting of amounts of the target compound from fly larvae in the first population of fly larvae.

### 8.3.1 Multiple Treatment Cycles

In one variation, a series of treatment cycles can be executed during the treatment period. In this variation, the population of fly larvae can be subjected to multiple treatment cycles (e.g., 2 treatment cycles, 3 treatment cycles, 5 treatment cycles) during the treatment period in order to increase enrichment of the target compound within the population of fly larvae. For example, during a treatment period, a first dosage (e.g., a 12-degree temperature increase applied over 45 minutes) of a first stressor (e.g., heat-shock stressor) can be applied to the population of fly larvae over a first treatment cycle within the treatment period. Additionally, the first treatment cycle can also include a first rest period following application of the first dosage of the first stressor, during which the population of fly larvae can be returned to the set of growth conditions of the growth period. After this first rest period, a second dosage (e.g., approximating the first dosage, greater than the first dosage) can be applied to the population of fly larvae over a second treatment cycle within the treatment period. Similarly, the second treatment cycle can include a second rest period. Additionally, the treatment period can include a third treatment cycle, a fourth treatment cycle, and so on, in order to increase enrichment of the target compound. Therefore, the treatment period can include a particular quantity of treatment cycles configured to maximize enrichment of the target compound within the population of fly larvae.

In one implementation, Blocks of the method S100 recite: during a first treatment cycle within the treatment period, applying a first dosage of a first stressor to the population of fly larvae; and, during a second treatment cycle succeeding the first treatment cycle within the treatment period, applying a second dosage of the first stressor—exceeding the first dosage—to the population of fly larvae to trigger production of the target compound.

In this implementation, the population of fly larvae—contained in the incubator cage 152 within the treatment module 130 (e.g., a treatment chamber) during the treatment period—can be exposed to stressor doses of increasing magnitude over successive treatment cycles in order to build tolerance to the stressor within the population of fly larvae. For example, during the incubation period, a population of fly larvae can be cultivated according to a set of incubator conditions defining a first incubation temperature range, such as between 25 degrees Celsius and 27 degrees Celsius. Then, during a first treatment cycle, within a treatment period succeeding the incubation period, the population of fly larvae can be located within a treatment chamber and held at temperatures—within the treatment chamber—within a second temperature range (e.g., between 30 degrees Celsius and 35 degrees Celsius, between 35 degrees Celsius and 37 degrees Celsius), temperatures within the second temperature range exceeding temperatures within the first temperature range. Then, during a second treatment cycle, succeeding the first treatment cycle within the treatment period, the population of fly larvae can be located (or maintained) within the treatment chamber and held at temperatures within a third temperature range (e.g., between 35 degrees Celsius and 37 degrees Celsius, between 37 degrees Celsius and 40 degrees Celsius), temperatures within the third temperature range exceeding temperatures within the second temperature range.

Additionally and/or alternatively, in another implementation, a rate of production and/or an amount of the target compound produced can be controlled via the dosage of the stressor applied to the population of fly larvae. However, if the dosage of the stressor is increased too quickly, the population of fly larvae may not survive application of the stressor. Therefore, a lower dosage of the stressor can initially be applied during a first treatment cycle, followed by a higher dosage during a subsequent treatment cycle, thus increasing tolerance of the insects to the stressor and enabling application of increased dosages of the stressor for increased enrichment of the target compound within the population of fly larvae.

In particular, in this implementation, during a treatment period: during a first treatment cycle within the treatment period, a first dosage of a first stressor can be applied to the population of fly larvae to trigger production of the first target compound in the population of fly larvae at a first rate proportional the first dosage; and, during a second treatment cycle within the treatment period, a second dosage, exceeding the first dosage, of the first stressor can be applied to the population of fly larvae to trigger production of the first target compound in the population of fly larvae at a second rate proportional the second dosage and exceeding the first rate.

### 8.4 Harvesting Larvae+Compound Extraction

Block S150 of the method S100 recites: in response to expiration of a duration of the treatment period, harvesting the first population of larvae from the second cage (e.g., the incubator cage 152) and extracting an amount of the first target compound from the first population of larvae.

Generally, in response to completion of the treatment period (e.g., as described above), the incubator cage 152—including a first population of fly larvae—can be harvested from the incubator cage 152 in preparation for extraction of amounts of the first target compound from fly larvae in the first population of fly larvae. For example, the first population of fly larvae can be: collected—in the incubator cage 152—from the treatment chamber of the treatment module 130; removed from the incubator cage 152; and mechanically homogenized—such as in a particular buffer matched to the first target compound—to form a homogenous mixture from which the target compound can be readily extracted, such as via implementation of affinity chromatography and/or size exclusion chromatography techniques.

### 8.4.1 Predicted Amount of the Target Compound

In one variation, the first population of fly larvae can be harvested in response to a predicted amount of the first target compound—produced by the first population of fly larvae in the treatment module 130 during the treatment period—exceeding a threshold amount defined for the first target compound. In particular, in this variation, Blocks of the method S100 can include: predicting a first amount (e.g., concentration, quantity, proportion) of the first target compound produced by fly larvae in the first population of fly larvae during the treatment period; and, in response to the first amount exceeding a threshold amount, harvesting the first population of fly larvae. Alternatively, in response to the first amount (e.g., a predicted amount) of the first target compound falling below the threshold amount, a subsequent treatment cycle—such as including application of a particular stressor within the treatment chamber—can be implemented in order to trigger further production of the first target compound in the first population of fly larvae.

In one implementation, an amount of the first target compound produced by fly larvae in the first population of fly larvae can be predicted based on detection of an optical signal generated by fly larvae, in the first population of fly larvae, responsive to production of the first target compound. In this implementation, a genome of a first fly population—and of the first population of fly larvae descending from the first fly population—can be genetically modified to include: a first promoter sequence; a first target sequence encoding for the first target compound and configured to express responsive to expression of the first promoter sequence; and a first reporter sequence linked to the first target sequence and encoding for a reporter protein configured to generate a detectable signal responsive to expression of the first target sequence. Then, in one example, during the treatment period for this first population of fly larvae, the computer system and/or controller 160—such as autonomously and/or in combination with a human user—can: access an image—captured by an optical sensor coupled to the treatment module 130—of fly larvae in the incubator cage 152; estimate a first amount of the first target compound generated by the first population of larvae based on a set of features extracted from the image; and, in response to the first amount exceeding a threshold amount, trigger harvesting of the first population of larvae from the incubator cage 152. For example, the genome of the fly population—and of the first population of fly larvae—can be genetically modified to include a first reporter sequence encoding for a fluorescent protein and configured to generate a detectable, fluorescence signal responsive to expression. The computer system and/or controller 160 can then estimate the first amount of the first target compound generated by the first population of larvae based on fluorescence measurements—such as an intensity of fluorescence—extracted from the image.

### 8.5 Growth Period: Replacing the Feeder Population

Block S160 of the method S100 recites: in response to expiration of a global duration defined for the first fly population in the feeder cage 150, harvesting the first fly population from the feeder cage 150 and depositing a second fly population—genetically modified to produce amounts of the first target compound—into the feeder cage 150 in replacement of the first fly population.

In particular, at an initial time, the first fly population (or “population of adult flies”) can be deposited into a first feeder cage 150 in the feeder module 110, and a timer for a fixed duration (e.g., one week, two weeks, one month)—defined for the first fly population—can be initiated. Then, populations of eggs can be extracted from the first fly population during a series of feeding cycles, and each population of eggs—derived from the first fly population—can be transferred to an incubator cage 152 in the incubator module 120, as described above.

Then, in response to expiration of the timer, an incubator cage 152—containing a population of fly larvae grown from a population of eggs derived from the first fly population—in the incubator module 120 can be transferred to a repopulation module 140 configured to promote transformation of the fly larvae in the population of fly larvae into a second fly population of adult flies. In particular, during a growth period of a fixed duration (e.g., 10 days, 14 days), the population of fly larvae can be: transferred to a cage—including a layer of foodstuff and a set of dividers extending from the layer of foodstuff—in the repopulation module 140; and subjected to a set of environmental conditions (e.g., a target air temperature, a target relative humidity) configured to promote transformation of the population of fly larvae into a second fly population (e.g., a second population of adult flies). Then, in response to expiration of the fixed duration of the growth period, the first fly population—in the first feeder cage 150 in the feeder module 110—can be extracted from the first feeder cage 150 and the second fly population can be deposited into the first feeder cage 150 in replacement of the first fly population. The second fly population can therefore replace the first fly population prior to expiration of a predicted lifespan of flies in the first fly population and/or prior to an expected decline in fly health—such as characterized by disease, fitness, quantity and/or quality of eggs deposited, fitness, etc.—thereby enabling continuous production of the first target compound while minimizing reductions in a rate of production of the first target compound, such as due to death and/or decline in health of flies in the first fly population.

### 9. Automated Facility

In one implementation, the system 100 can be distributed throughout an automated facility configured to enable autonomous execution of Blocks of the method S100. In particular, in this implementation, the system 100 can include: a local or remote controller 160, such as a remote server or a remote computer system; a robotic manipulator 190 (e.g., a robotic arm) coupled to the controller 160, located at a transfer station within the facility, and outfitted with a camera, scale, and/or end effector configured to retrieve cages (e.g., feeder, incubator, and/or repopulation cages 154) from modules; and/or an automated vehicle coupled to the controller 160 and configured to retrieve modules from defined regions throughout the facility, to deliver modules to the transfer station, and to return modules to their assigned regions throughout the facility.

In the preceding example, the system 100 can further include a vehicle configured to: autonomously navigate throughout the facility; autonomously deliver the first feeder module 110 from the first region to the transfer station; and autonomously deliver the incubator module 120 to the transfer station. In this example, the system 100 can further include a robotic manipulator 190 arranged at the transfer station and configured to: engage a first feeder cage 150, in a first set of feeder cages 150, occupying a first feeder cage slot in the first array of feeder cage slots, in the first feeder module 110; disassemble the first feeder cage 150 to separate a first food container 156 from a first tray 158—containing flies in the dormant state—of the first feeder cage 150; reassemble the first feeder cage 150 by locating a second food container 156 over the first tray 158; insert the first feeder cage 150 into the first feeder cage slot in the first set of slots, in the first feeder module 110, in the inverted position; assemble a first incubator cage 152 by locating a second tray 158 over the first food container 156; insert the first incubator cage 152 into a first incubator cage slot, in the array of incubator cage slots, in the incubator module 120; and repeat this process for each feeder cage 150 in the first set of feeder cages 150. The vehicle can therefore further be configured to: autonomously return the first feeder module 110 from the transfer station to the first region upon reloading of the first set of feeder cages 150 with new food containers 156; and autonomously return the incubator module 120 from the transfer station to the second region upon loading of the set of incubator cages 152 into the incubator module 120. Alternatively, a user may manually redistribute modules throughout the facility and/or manually retrieve and replace fly cages within these modules.

In one implementation, the automated facility can include: one or more feeder areas configured to receive a set of feeder modules 110—loaded with feeder cages 150—during egg-laying periods for these feeder modules 110; one or more incubator areas configured to receive the set of incubator modules 120—loaded with incubator cages 152—during incubation periods for these incubator modules 120; a repopulation area configured to receive a set of repopulation modules 140—loaded with repopulation cages 154—during repopulating periods for these repopulation modules 140; a treatment chamber configured to receive the incubator module 120 and facilitate heat-shock treatment of larvae contained in the incubator cages 152; a harvest station configured to facilitate harvesting of larvae from incubator cages 152 contained in the incubator module 120. The automated facility can further include a set of transfer stations, such as: a food transfer station configured to facilitate transfer of food containers 156 from the feeder module 110 to the incubator module 120; a larvae transfer station configured to facilitate transfer of larvae extracted from the incubator cages 152 into the repopulation cages 154; and/or a reloading station configured to facilitate transfer of replacement adult-fly populations into feeder cages 150 within the feeder module 110.

In one example, the automated facility can include: a first feeder area configured to receive a first set of feeder modules 110 transiently loaded with feeder cages 150 containing populations of adult flies of a first type genetically modified to produce amounts of a first target compound; and a second feeder area configured to receive a second set of feeder modules 110 transiently loaded with feeder cages 150 containing populations of adult flies of a second type genetically modified to produce amounts of a second target compound. The automated facility can further include additional feeder areas—such as a third feeding area, a fourth feeding area, etc.—configured to receive feeder modules 110 assigned to populations of adult flies of varying types, thereby enabling physical separation of flies of different types and therefore minimizing cross-contamination of these flies. The automated facility can similarly include distinct incubator areas, repopulation areas, and/or treatment areas for offspring (e.g., eggs, larvae, flies) of adult flies of different types.

In this implementation, the system 100 can therefore automatically: collect and transfer modules throughout the facility according to a defined rearing schedule; and regulate ambient environmental conditions within these areas and/or modules—via environmental controls arranged in these areas and/or modules—to cultivate populations of adult flies and/or fly larvae and promote generation of the target compound within these populations.

In particular, in one example, the system 100 can include: a suite of sensors—configured to output signals representing environmental conditions at the set of modules—including a first set of sensors 170 coupled to the feeder module 110, a second set of sensors 170 coupled to the incubator module 120, a third set of sensors 170 coupled to the treatment module 130, and a fourth set of sensors 170 coupled to the repopulation module 140; and a suite of environmental controls—configured to regulate environmental conditions within the facility—including a first set of environmental controls 180 installed in the first region, a second set of environmental controls 180 installed in the second region, a third set of environmental controls 180 installed in the third region, and a fourth set of environmental controls 180 installed in the fourth region. The system 100 can further include a controller 160 configured to: read a first set of signals from the first set of sensors 170; selectively trigger actuation of the first set of environmental controls 180 to regulate environmental conditions at the feeder module 110 according to a target set of feeder conditions defined for the feeder module 110 and configured to promote deposition of eggs by adult flies in feeder cages 150; read a second set of signals from the second set of sensors 170; selectively trigger actuation of the second set of environmental controls 180 to regulate environmental conditions at the incubator module 120 according to a target set of incubator conditions defined for the incubator module 120 and configured to promote transformation of fly eggs into fly larvae in incubator cages 152; read a third set of signals from the third set of sensors 170; selectively trigger actuation of the third set of environmental controls 180 to regulate environmental conditions at the treatment module 130 according to a target set of treatment conditions defined for the treatment module 130 and configured to promote generation of the first target compound in fly larvae; read a fourth set of signals from the fourth set of sensors 170; and selectively trigger actuation of the fourth set of environmental controls 180 to regulate environmental conditions at the repopulation module 140 according to a target set of growth conditions defined for the repopulation module 140 and configured to promote growth of fly larvae into adult flies.

For example, the system 100 can interface with a set of sensors 170 and a set of environmental controls 180 installed throughout the automated facility to: regulate temperature within the feeder area within a threshold deviation of 25 degrees Celsius; regulate relative humidity within the feeder area within a threshold deviation of approximately 70 percent; regulate temperature within the incubator area according to a target incubator temperature range—such as at approximately (e.g., within five percent) 25 degrees Celsius during a first time period and at approximately 27 degrees Celsius over a second time period—during an incubation period of a target duration (e.g., 65 hours) defined for the incubator module 120; regulate relative humidity within the incubator area within a target humidity range between 70 percent and 85 percent; regulate temperature within the treatment chamber according to a target treatment temperature range—such as increasing from 27 degrees Celsius to 40 degrees Celsius over an 8-hour period—defined for the treatment period; and regulate relative humidity within the treatment chamber within a threshold deviation of approximately 80 percent.

In one example, the system 100 can interface with the set of sensors 170 and the set of environmental controls 180 to: regulate temperature within the feeder area at a target feeder temperature of approximately (e.g., within 5 percent) 25 degrees Celsius and thus maintain the set of feeder modules within the feeder area at approximately the target feeder temperature for a duration of a feeding period; regulate temperature within the incubator area at a target incubator temperature of approximately 27 degrees Celsius and thus heat the set of incubator modules—loaded with incubator cages including food containers extracted from feeder modules in the feeder area—from an initial temperature of approximately 25 degrees Celsius to a final temperature of approximately 27 degrees Celsius and maintain the set of incubator modules at approximately 27 degrees Celsius for a remaining duration of an incubator period; and regulate temperature within the treatment chamber at a target treatment temperature of approximately 40 degrees Celsius and thus heat the set of incubator cages—extracted from the set of incubator modules in the incubator area—from an initial temperature of approximately 27 degrees Celsius to a final temperature of approximately 40 degrees Celsius and maintain the set of incubator cages at approximately 40 degrees Celsius for a remaining duration of a treatment period.

### 9.1 Yield Model

In one variation, the computer system can derive a yield model configured to predict yield, of the target compound, for a particular fly population and/or larvae population derived from the fly population, such as based on ambient conditions (e.g., humidity, temperature, pressure) within the modules during rearing of these populations.

In particular, in this variation, the method S100 can be executed by the computer system in conjunction with the automated facility: to autonomously collect ambient data—such as including humidity data, temperature data, pressure data, etc.—from these modules and/or near these modules over time through a combination of fixed and mobile high- and low-resolution ambient sensors; to collect population outcome data, such as size, weight, survival rate, number of eggs laid, number of offspring, energy level, food consumption rate, visual characteristics, for all or select fly populations and/or larvae populations; to collect yield outcome data, such as an amount of the target compound generated by a particular larvae population and/or by offspring (e.g., larvae) of a particular fly population; to amass these ambient and outcome data in population-specific data containers (e.g., discrete files or vectors); and to derive a model for predicting outcomes for a singular fly population—such as an amount of the target compound generated by all offspring of this fly population during a life cycle for this fly population—and/or a larvae population at harvest (and during growth cycles) based on ambient conditions during rearing of the fly population and/or larvae population and/or qualities of these populations recorded hours, days, or weeks before the population is harvested.

The computer system can implement this model to automatically define or adjust growth input parameters realized autonomously by systems throughout the facility while growing new fly and/or larvae populations in order to drive these new populations toward greater yield, reduced cost, reduced population loss (e.g., due to improved growth conditions and/or mitigation procedures), etc. The system 100 can then autonomously implement these adjusted growth input parameters (e.g., in a revised “rearing schedule”) for new populations grown at the facility. The system 100 can then repeat these processes over time to collect fly and/or larvae maturation data for these new populations, to collect yield outcome data for these new populations, to refine the yield model, and to revise growth input parameters for subsequent populations of fly and/or larvae populations reared at the facility.

The system 100 can therefore: aggregate input data—such as air humidity, temperature, pressure, air flow, etc.—and output data across a group of fly and larvae populations (e.g., hundreds or thousands of populations); implement machine learning, artificial intelligence, and/or other statistical techniques (e.g., regression analysis, least square error analysis) to derive correlations between input parameters and outcomes for these populations; represent these correlations in a model; define target values and tolerance ranges for various input parameters based on correlations with various outcomes represented in this model; and generate a temporal rearing schedule that, when implemented for new fly and larvae populations reared in the facility, is predicted to yield certain target outcomes (e.g., target yield for the target compound, target time to harvest, target survival rate).

For example, the computer system can: access a corpus of timeseries environmental data, captured by a set of sensors 170 installed within the facility at the set of modules, representing ambient environmental conditions at the set of modules during the initial time period; access a corpus of timeseries amounts of the target compound extracted from fly larvae during the initial time period; derive a model linking amounts of the target compound generated by larvae populations to a set of environmental conditions at the set of modules; and—based on the model and a target amount of the target compound defined for the global period and/or for a particular fly population—define a first set of environmental conditions for the feeder module 110, a second set of environmental conditions for the incubator module 120, a third set of environmental conditions for the treatment module 130, and/or a fourth set of environmental conditions for the repopulation module 140.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method comprising:
during a first feeding period of a first fixed duration:
at a first time, coupling a first container to a first tray, occupied by a first population of adult flies, to form a first cage, in a first set of cages, arranged in an inverted configuration and configured to contain the first population of adult flies, the first container loaded with a layer of foodstuff facing and configured to seat over the first tray with the layer of foodstuff facing an interior volume of the first tray in the inverted configuration, the first population of adult flies genetically modified to produce amounts of a first target compound;
subjecting the first set of cages, arranged within a feeder module, to a first set of conditions configured to promote deposition of fly eggs by adult flies; and
at a second time succeeding the first time by the first fixed duration, decoupling the first container from the first tray to collect a first population of eggs deposited onto the first container by the first population of adult flies during the first feeding period;
during a first incubation period, succeeding the first feeding period, of a second fixed duration:
at a third time, coupling the first container to a second tray to form a second cage, in a second set of cages, arranged in an upright configuration, the second tray configured to seat over the first container in the upright configuration and the first container including the first population of eggs facing an interior volume of the second tray at the third time; and
subjecting the second set of cages, arranged within an incubation module, to a second set of conditions configured to promote transformation of fly eggs into fly larvae;
in response to expiration of the second fixed duration, during a first treatment period succeeding the first incubation period and of a third fixed duration, locating the second cage in a treatment module configured to promote generation of the target compound in fly larvae, the second cage containing a first population of larvae grown from the first population of eggs during the first incubation period; and
in response to expiration of the third fixed duration:
harvesting the first population of larvae from the second cage; and
extracting an amount of the first target compound from the first population of larvae.

2. The method of claim 1:
wherein subjecting the first set of cages arranged within the feeder module to the first set of conditions configured to promote deposition of eggs by adult flies comprises:
regulating a temperature of the feeder module within a first target deviation of 25 degrees Celsius; and
regulating a relative humidity of the feeder module within a second target deviation of 70 percent relative humidity; and
wherein subjecting the second set of cages arranged within the incubation module to the second set of conditions configured to promote transformation of fly eggs into fly larvae comprises:
at a first time, regulating a temperature of the incubation module within a third target deviation of 25 degrees Celsius;
at a second time succeeding the first time, regulating the temperature of the incubation module within a fourth target deviation of 27 degrees Celsius; and
regulating a relative humidity of the incubation module within a target humidity range of 70 percent relative humidity to 85 percent relative humidity.

3. The method of claim 1, further comprising:
during an initial time period:
depositing the first population of adult flies into the first tray; and
initiating a timer for a global duration; and
during a second cycle:
during a second feeding period of the first fixed duration:
at a fourth time, coupling a second container to the first tray occupied by the first population of adult flies to form a third cage arranged in the inverted configuration and configured to contain the first population of adult flies, the second container loaded with a second layer of foodstuff and configured to seat over the tray with the second layer of foodstuff facing the interior volume of the tray in the inverted configuration; and
at a fifth time succeeding the fourth time by the first fixed duration, decoupling the second container from the first tray to collect a second population of eggs deposited onto the second container by the first population of adult flies during the second feeding period;
during a second incubation period succeeding the second feeding period and of a third fixed duration:
coupling the second container to a third tray to form a third cage arranged in the upright configuration, the third tray configured to seat over the second container in the upright configuration, the second container including the population of eggs facing an interior volume of the third tray; and
subjecting the third cage to the second set of conditions;
in response to expiration of the third fixed duration and in response to expiration of the global duration, during a growth period succeeding the second incubation period and of a fourth fixed duration:
decoupling the second container from the third tray to collect the second population of larvae grown from the population of eggs during the second incubation period; and
coupling the second container to a fourth tray to form a third cage arranged in the upright configuration, the fourth tray:
configured to seat over the second container in the upright configuration with the second population of larvae facing an interior volume of the fourth tray;
including a second layer of foodstuff arranged across a surface of the fourth tray facing and opposite the second container; and
including a set of dividers extending from the second layer of foodstuff; and
subjecting the third cage to a third set of conditions configured to promote transformation of the second population of larvae into a second population of adult flies; and
in response to expiration of the fourth fixed duration:
extracting the first population of adult flies from the first tray; and
depositing the second population of adult flies into the first tray.

4. The method of claim 1, further comprising, during an initial time period, genetically modifying a genome of the first population of adult flies to include:
a target sequence encoding for the first target compound; and
a promoter sequence coupled to the target sequence, expression of the promoter sequence configured to trigger expression of the target sequence.

5. The method of claim 4:
wherein genetically modifying the genome of the first population of adult flies to include the promoter sequence comprises genetically modifying the genome of the first population of adult flies to include the promoter sequence associated with a first stressor; and
wherein locating the second cage in the treatment chamber configured to promote generation of the target compound comprises:
locating the second cage in the treatment chamber; and
applying the first stressor to larvae in the second cage, within the treatment chamber, to trigger expression of the promoter sequence and induce generation of the first target compound via expression of the target sequence responsive to expression promoter sequence.

6. The method of claim 1, wherein locating the second cage in the treatment chamber configured to promote generation of the target compound comprises:
locating the second cage in the treatment chamber;
over a first treatment cycle, applying a first dosage of a first stressor to larvae in the second cage, within the treatment chamber, to trigger production of the first target compound at a first rate corresponding to the first dosage; and
over a second treatment cycle succeeding the first treatment cycle, applying a second dosage of the first stressor to larvae in the second cage, within the treatment chamber, to trigger production of the first target compound at a second rate corresponding to the second dosage and exceeding the first rate, the second dosage exceeding the first dosage.

7. The method of claim 6:
wherein applying the first dosage of the first stressor to larvae in the second cage over the first treatment cycle comprises applying the first dosage of a heat-shock stressor to larvae in the second cage comprising heating the treatment chamber to temperatures within a first target temperature range over the first treatment cycle; and
wherein applying the second dosage of the first stressor to larvae in the second cage over the second treatment cycle comprises applying the second dosage of the heat-shock stressor to larvae in the second cage comprising heating the treatment chamber to temperatures within a second target temperature range over the second treatment cycle, temperatures in the second target temperature range exceeding temperatures in the first target temperature range.

8. The method of claim 1:
wherein subjecting the first set of cages arranged within the feeder module to the first set of conditions configured to promote deposition of eggs by adult flies comprises subjecting the first set of cages arranged within the feeder module to the first set of conditions configured to promote deposition of eggs by adult flies, the first set of cages comprising the first cage and a third cage occupied by a second population of adult flies genetically modified to produce amounts of a second target compound; and
further comprising:
during the first feeding period:
at approximately the first time, coupling a second container to a third tray, occupied by the second population of adult flies, to form the second cage arranged in the inverted configuration, the second container loaded with a second layer of foodstuff facing and configured to seat over the third tray with the second layer of foodstuff facing an interior volume of the third tray in the inverted configuration; and
at approximately the second time, decoupling the second container from the third tray to collect a second population of eggs deposited onto the second container by the second population of adult flies during the first feeding period;
during the first incubation period, at approximately the third time, coupling the second container to a fourth tray to form a fourth cage arranged in the upright configuration, the fourth tray configured to seat over the second container in the upright configuration and the second container including the second population of eggs facing an interior volume of the fourth tray;
in response to expiration of the second fixed duration, during the first treatment period, locating the fourth cage in the treatment chamber; and
in response to expiration of the third fixed duration:
harvesting the second population of larvae from the fourth cage; and
extracting a second amount of the second target compound from the second population of larvae.

9. The method of claim 8, wherein subjecting the first set of cages arranged within the feeder module to the first set of conditions, the first set of cages comprising the first cage and the third cage comprises subjecting the first set of cages arranged within the feeder module to the first set of conditions, the first set of cages comprising:
the first cage occupied by the first population of adult flies genetically modified to produce amounts of the first target compound comprising a growth factor; and
the third cage occupied by the second population of adult flies genetically modified to produce amounts of the second target compound comprising an insulin protein.

10. The method of claim 1:
wherein harvesting the first population of larvae from the second cage comprises harvesting the first population of larvae from the second cage at a fourth time; and
further comprising:
during a second feeding period succeeding the first feeding period and of the first fixed duration:
subjecting the first set of cages arranged within the feeder module to the first set of conditions;
at a fifth time, coupling a second container to the first tray, occupied by the first population of adult flies, to form the first cage arranged in the inverted configuration, the second container loaded with a second layer of foodstuff facing and configured to seat over the first tray with the second layer of foodstuff facing the interior volume of the first tray in the inverted configuration; and
at a sixth time succeeding the fourth time by the first fixed duration, decoupling the second container from the first tray to collect a second population of eggs deposited onto the second container by the first population of adult flies during the second feeding period; and
during a second incubation period, succeeding the second feeding period, of the second fixed duration:
coupling the second container to a third tray to form a third cage arranged in the upright configuration, the third tray configured to seat over the second container in the upright configuration and the second container including the second population of eggs facing an interior volume of third second tray; and
subjecting the third cage to the second set of conditions configured to promote transformation of the second population of eggs into a third population of larvae;
in response to expiration of the second fixed duration, during a second treatment period, succeeding the second incubation period, of the third fixed duration, locating the third cage in the treatment chamber; and
in response to expiration of the third fixed duration:
harvesting the second population of larvae from the third cage at a seventh time succeeding the fourth time by approximately the first fixed duration; and
extracting a second amount of the first target compound from the second population of larvae.

11. The method of claim 1, wherein coupling the first container to the first tray to form the first cage comprises coupling the first container to the first tray to form the first cage:
populated by the first population of adult flies;
defining a standard cage size configured to nest within a cage slot, in a set of cage slots within the feeder module, of a standard slot size; and
comprising:
the first container defining:
a base surface;
a set of container walls extending from the base surface and defining a first height;
a container rim extending from the set of container walls, opposite the base surface, and defining a first set of coupling features configured to transiently engage coupling features of a set of trays comprising the first tray; and
a set of ramps extending upward from the base surface toward the container rim; and
the first tray defining:
a food surface;
the layer of foodstuff arranged on the food surface, configured for consumption by the first population of adult flies, and configured to receive and retain eggs laid by adult flies in the first population of adult flies;
a set of tray walls extending from a perimeter of the food surface and defining a second height; and
a tray rim extending from the set of tray walls opposite the food surface and defining a second set of coupling features configured to transiently couple to the first set of coupling features to form the first cage.

12. The method of claim 1:
wherein subjecting the first set of cages, arranged within the feeder module, to the first set of conditions configured to promote deposition of fly eggs by adult flies comprises:
receiving a first set of signals from a first set of sensors coupled to the feeder module, the first set of signals representing air temperature and air pressure at the feeder module; and
based on the first set of signals:
selectively actuating a first temperature regulator installed at the feeder module to regulate a first air temperature at the feeder module according to a target air temperature defined by the first set of conditions; and
selectively actuating a first pressure regulator installed at the feeder module to regulate a first air pressure at the feeder module according to a target air temperature defined by the first set of conditions; and
wherein subjecting the second set of cages, arranged within the incubator module, to the second set of conditions comprises:
receiving a second set of signals from a second set of sensors coupled to the incubator module, the second set of signals representing air temperature and air pressure at the incubator module; and
based on the second set of signals:
selectively actuating a second temperature regulator installed at the incubator module to regulate a second air temperature at the incubator module according to a target air temperature defined by the second set of conditions; and
selectively actuating a second pressure regulator installed at the incubator module to regulate a second air pressure at the incubator module according to a target air temperature defined by the second set of conditions.

13. The method of claim 1:
wherein coupling the first container to the first tray occupied by the first population of adult flies genetically modified to produce amounts of the first target compound comprises coupling the first container to the first tray occupied by the first population of adult flies genetically modified to include:
a first promoter sequence;
a first target sequence encoding for the first target compound and configured to express responsive to expression of the first promoter sequence; and
a first reporter sequence linked to the first target sequence and encoding for a reporter protein configured to generate a detectable signal responsive to expression of the first target sequence; and
wherein harvesting the first population of larvae from the second cage in response to expiration of the third fixed duration comprises, in response to expiration of the third duration:
accessing an image of fly larvae in the second cage, the image captured by an optical sensor coupled to the treatment module;
estimating a first amount of the target compound generated by the first population of larvae based on a set of features extracted from the image; and
in response to the first amount exceeding a threshold amount, harvesting the first population of larvae from the second cage.

14. The method of claim 13:
wherein coupling the first container to the first tray occupied by the first population of adult flies genetically modified to include the first reporter sequence linked to the first target sequence and encoding for the reporter protein configured to generate the detectable signal comprises coupling the first container to the first tray occupied by the first population of adult flies genetically modified to include the first reporter sequence linked to the first target sequence and encoding for the reporter protein configured to generate the detectable signal comprising a fluorescence signal, the reporter protein comprising a fluorescent protein; and
wherein estimating the first amount of the target compound generated by the first population of larvae based on the set of features extracted from the image comprises estimating the first amount of the target compound generated by the first population of larvae based on an intensity of fluorescence depicted in the image.

15. A method comprising:
at a feeder module defining a first array of cage slots, subjecting a first set of cages, transiently arranged within first array of cage slots, to a first set of environmental conditions configured to promote deposition of fly eggs by adult flies, each cage, in the first set of cages, containing a fly population, in a set of fly populations, genetically modified to generate amounts of a target compound;
at an incubator module defining a second array of cage slots, subjecting a second set of cages, transiently arranged within second array of cage slots, to a second set of environmental conditions configured to promote transformation of fly eggs into fly larvae;
at a treatment module defining a third array of cage slots, subjecting a third set of cages, transiently arranged within third array of cage slots, to a third set of environmental conditions configured to promote generation of a target compound in fly larvae contained within the third set of cages;
at a repopulation module defining a fourth array of cage slots, subjecting a fourth set of cages, transiently arranged within fourth array of cage slots, to a fourth set of environmental conditions configured to promote transformation of fly larvae into adult flies; and
during a global period of a first target duration:
during a first cycle:
at a first time, transferring a first population of eggs, deposited by a first fly population in a first cage in the first set of cages, from the first cage to a second cage, in the second set of cages, in the incubator module;
at a second time succeeding the first time by a second target duration, transferring the second cage from the incubator module to the treatment module, the second cage containing a first population of larvae, emerged from the first population of eggs, at the second time; and
at a third time succeeding the second time by a third target duration, harvesting the first population of larvae from the second cage for extraction of an amount of the target compound; and
during a second cycle:
at a fourth time, transferring a second population of eggs, deposited by adult flies in the first fly population in the first cage, from the first cage to a third cage, in the second set of cages, in the incubator module;
at a fifth time succeeding the first time by the second target duration, in response to expiration of the first target duration, transferring the third cage from the incubator module to the repopulation module, the third cage containing a second population of larvae, emerged from the second population of eggs, at the fifth time; and
at a sixth time succeeding the fifth time by a fourth target duration:
harvesting the first fly population from the first cage; and
depositing a second fly population, grown from the second population of larvae in the repopulation module, in the first cage in the feeder module.

16. The method of claim 15:
further comprising, during a first feeding period preceding the first time, coupling a first container to a first tray, occupied by the first fly population, to form the first cage arranged in an inverted configuration in the feeder module and configured to contain the first fly population, the first container loaded with a layer of foodstuff facing and configured to seat over the first tray with the layer of foodstuff facing an interior volume of the first tray in the inverted configuration; and
wherein transferring the first population of eggs, deposited by the first fly population, from the first cage to the second cage in the incubator module comprises:
decoupling the first container from the first tray to collect the first population of eggs deposited onto the first container by the first fly population during the first feeding period; and
coupling the first container to a second tray to form the second cage arranged in an upright configuration in the incubator module, the second tray configured to seat over the first container in the upright configuration and the first container including the first population of eggs facing an interior volume of the second tray.

17. The method of claim 15, further comprising:
at a second feeder module defining a fifth array of cage slots, subjecting a fifth set of cages, transiently arranged within the fifth array of cage slots, to a fifth set of environmental conditions configured to promote deposition of fly eggs by adult flies, each cage, in the first set of cages, containing a fly population, in a second set of fly populations, genetically modified to generate amounts of a second target compound;
at a second incubator module defining a sixth array of cage slots, subjecting a sixth set of cages, transiently arranged within a sixth array of cage slots, to a sixth set of environmental conditions configured to promote transformation of fly eggs into fly larvae;
at a third treatment module defining a seventh array of cage slots, subjecting a seventh set of cages, transiently arranged within a seventh array of cage slots, to a seventh set of environmental conditions configured to promote generation of the second target compound in fly larvae contained within the seventh set of cages;
at a second repopulation module defining an eighth array of cage slots, subjecting an eighth set of cages, transiently arranged within an eighth array of cage slots, to an eighth set of environmental conditions configured to promote transformation of fly larvae into adult flies; and
during a second global period of a second target duration:
during a third cycle:
at a seventh time, transferring a third population of eggs, deposited by adult flies of a second fly population in a first cage, in the fifth set of cages, from the first cage to a second cage, in the sixth set of cages, in the second incubator module;
at an eighth time succeeding the seventh time by a fifth target duration, transferring the second cage from the second incubator module to the second treatment module, the second cage containing a third population of larvae, emerged from the third population of eggs, at the eighth time; and
at a ninth time succeeding the eighth time by a sixth target duration, harvesting the third population of larvae from the second cage for extraction of an amount of the second target compound.

18. The method of claim 15, further comprising, during an initial time period preceding the global period:
accessing a corpus of timeseries environmental data, captured by a set of sensors installed within the facility at the set of modules, representing ambient environmental conditions at the set of modules during the initial time period;
accessing a corpus of timeseries amounts of the target compound extracted from fly larvae during the initial time period;
deriving a model linking amounts of the target compound generated by larvae populations to a set of environmental conditions at the set of modules; and
based on the model and a target amount of the target compound defined for the global period, defining the first set of environmental conditions for the feeder module, the second set of environmental conditions for the incubator module, the third set of environmental conditions for the treatment module, and the fourth set of environmental conditions for the repopulation module.

19. A system comprising:
a set of modules comprising:
a feeder module transiently arranged in a first region of a facility and defining a first array of cage slots assigned to feeder cages containing adult fly populations genetically modified to generate amounts of a first target compound;
an incubator module transiently arranged in a second region of the facility and defining a second array of cage slots configured to receive incubator cages containing populations of fly eggs configured to transform into populations of fly larvae;
a treatment module transiently arranged in a third region of the facility and defining a third array of cage slots configured to receive incubator cages containing populations of fly larvae; and
a repopulation module transiently arranged in a fourth region of the facility and defining a fourth array of cage slots configured to receive repopulation cages containing population of fly larvae configured to transform into populations of adult flies;
a suite of sensors configured to output signals representing environmental conditions at the set of modules and comprising a first set of sensors coupled to the feeder module, a second set of sensors coupled to the incubator module, a third set of sensors coupled to the treatment module, and a fourth set of sensors coupled to the repopulation module;
a suite of environmental controls configured to regulate environmental conditions within the facility and comprising a first set of environmental controls installed in the first region, a second set of environmental controls installed in the second region, a third set of environmental controls installed in the third region, and a fourth set of environmental controls installed in the fourth region;
a set of robotic manipulators configured to transiently retrieve cages from the set of modules, transfer cages between modules in the set of modules, and locate cages within the set of modules; and
a controller configured to:
coordinate motion of the set of robotic manipulators according to a schedule defined for fly populations genetically modified to generate the first target compound;
based on signals output by the first set of sensors, selectively trigger actuation of the first set of environmental controls to regulate environmental conditions at the feeder module according to a target set of feeder conditions defined for the feeder module and configured to promote deposition of eggs by adult flies in feeder cages;
based on signals output by the second set of sensors, selectively trigger actuation of the second set of environmental controls to regulate environmental conditions at the incubator module according to a target set of incubator conditions defined for the incubator module and configured to promote transformation of fly eggs into fly larvae in incubator cages;
based on signals output by the third set of sensors, selectively trigger actuation of the third set of environmental controls to regulate environmental conditions at the treatment module according to a target set of treatment conditions defined for the treatment module and configured to promote generation of the first target compound in fly larvae; and
based on signals output by the fourth set of sensors, selectively trigger actuation of the fourth set of environmental controls to regulate environmental conditions at the repopulation module according to a target set of growth conditions defined for the repopulation module and configured to promote growth of fly larvae into adult flies.

20. The system of claim 19:
wherein the feeder module defines the first array of cage slots assigned to feeder cages comprising a first feeder cage:
arranged in an inverted configuration within a first slot, in the first array of cage slots; and
comprising a first container transiently coupled to a first tray, in a set of trays, occupied by a first fly population, the first container loaded with a layer of foodstuff facing and configured to seat over the first tray with the layer of foodstuff facing an interior volume of the first tray in the inverted configuration; and
wherein the incubator module defines the second array of cage slots assigned to incubator cages comprising a first incubator cage:
arranged in an upright configuration within a first slot, in the second array of cage slots; and
comprising the first container transiently coupled to a second tray, in the set of trays, the second tray configured to seat over the first container in the upright configuration and the first container including a first population of eggs, deposited by the first fly population, facing an interior volume of the second tray.
